(19) United States
(12) Patent Application Publication
(10) Pub. No.: US 2018/0228430 A1
(43) Pub. Date: Aug. 16, 2018

(54) SYSTEM, METHOD AND APPARATUS FOR REHABILITATION WITH TRACKING

(71) Applicant: MindMaze Holding SA, Lausanne (CH)

(72) Inventors: Daniel PEREZ MARCOS, Lausanne (CH); Cyntia DUC, Lausanne (CH); Gangadhar GARIPELLI, Lausanne (CH); Tej TADI, Lausanne (CH); Solange SEPPEY, Lausanne (CH)

(21) Appl. No.: 15/893,637

(22) Filed: Feb. 11, 2018

Related U.S. Application Data
(60) Provisional application No. 62/457,192, filed on Feb. 10, 2017.
(60) Provisional application No. 62/467,872, filed on Mar. 7, 2017.

(51) Int. Cl. A61B 5/00; A61B 5/11; A61B 5/22; A61B 5/0482; G06F 3/01; G16H 40/63

(52) U.S. Cl. CPC A61B 5/4848; A61B 5/0077; A61B 5/1125; A61B 5/1128; A61B 5/742; A61B 5/4824; A61B 2505/09; A61B 5/0482; A61B 5/4884; A61B 5/1114; G06F 3/011; G16H 40/63; A61B 5/225

(57) ABSTRACT

A system, method and apparatus for rehabilitation with computational feedback, based upon tracking the movement of the user.

### FIELD OF THE INVENTION

The present invention is of a system, method and apparatus for rehabilitation with tracking, and in particular, to such a system, method and apparatus for rehabilitation with computational feedback, based upon tracking the movement of the user.

### BACKGROUND OF THE INVENTION

A stroke is a cerebrovascular accident that happens when the blood flow to a portion of the brain is disrupted, resulting in brain cell death. The consequences can be physical as well as cognitive, and can lead to a decrease in movement function and a loss of independence. This disorder is a major cause of long-term physical disabilities and handicaps in Western countries, mostly in the older age range of the population. Thus, as the worldwide population is aging, this disorder is one of the main concerns for the future of health care due to budgetary constraints limiting the intensity and length of the conventional rehabilitative treatment consisting of physical and occupational therapy (C. Bosecker et al., “Kinematic robot-based evaluation scales and clinical counterparts to measure upper limb motor performance in patients with chronic stroke,” Neurorehabilitation and Neural Repair, 2010).

Stroke survivors often experience hemiparesis, i.e., weakness on one side of the body, which can in turn lead to upper limb impairment and thus to a decrease in their independence and quality of life (I. Aprile et al., “Kinematic analysis of the upper limb motor strategies in stroke patients as a tool towards advanced neurorehabilitation strategies: A preliminary study,” BioMed Research International, 2014).

Stroke patients often use compensatory strategies to fill in for their lack of mobility, by using, for example, trunk recruitment, fixation of specific body segments or pathological synergies, e.g., reduction in shoulder movement can be compensated by gross flexion of the elbow, wrist moment and gravity (see, for example, M. C. Cirstea and M. F. Levin, “Compensatory strategies for reaching in stroke,” Brain, 2000; W. Liu et al., “Compensatory arm reaching strategies after stroke: Induced position analysis,” Journal of Rehabilitation Research and Development, 2013). M. C. Cirstea and M. F. Levin showed that the use of compensatory movement correlated well with the level of deficit and degree of spasticity of the patients. However, they also suggested that the use of inappropriate compensatory movements might have a negative impact on the rehabilitative process. Indeed, such strategies favor distorted positions of the joints, thus leading to a shortening of the muscles, which is an obstacle to recovery.

The motor deficits that may appear in the upper limb post-stroke are characterized by movements that show segmented patterns, meaning that they are composed of a concatenation of sub-movements (B. Rohrer et al., “Movement smoothness changes during stroke recovery,” The Journal of Neuroscience, 2002; L. van Dokkum et al., “The contribution of kinematics in the assessment of upper limb motor recovery early after stroke,” Neurorehabilitation and Neural Repair, 2013). This lack of smoothness together with slowness leads to a decrease in motivation for the use of the paretic arm (M. F. Levin et al., “Virtual reality environments to enhance upper limb functional recovery in patients with hemiparesis.” Studies in Health Technology and Informatics, 2009). Indeed, it causes clumsiness and a low level of precision, which make it difficult for the patients to perform Activities of Daily Living (ADL).

The difficulty of rehabilitation is increased, because these programs often have a low compliance if they are carried out without the presence of a therapist, due to a lack of motivation and slow evolution of the patient's progress. This is a problem because of the constant increase of cases and the lack of time for the therapist to fully be involved in everyone's therapy to the extent required.

### BRIEF SUMMARY OF THE INVENTION

The present invention, in at least some embodiments, is of a system, method and apparatus for rehabilitation with computational feedback, based upon tracking the movement of the user. Such a system, method and apparatus may be performed with or without the presence of therapist, increasing the therapeutic opportunities for the patient.

According to at least some embodiments, there is provided a method with a computationally directed set of movements, the set of movements being directed by a computational system that comprises providing visual direction to the patient and tracking the movements of the patient, the computational system comprising a display, at least one tracking sensor and a plurality of machine instructions for controlling the display to provide the visual direction and for receiving sensor data from the tracking sensor to track the movements of the patient, the method comprising: displaying a virtual object to the patient; indicating a movement to be performed with the virtual object; tracking said movement; and adjusting said displaying and said indicating according to said tracking; wherein said displaying, indicating and tracking apply a higher degree of therapeutic intensity as compared to a standard of care rehabilitative measure.

Optionally the computational system comprises: a depth camera and an RGB camera for obtaining tracking data, a tracking engine for tracking the movements of the patient, and a data analysis layer for analyzing the tracked movements and for adjusting said displaying and said indicating according to said tracking.

Optionally the computational system comprises a MindMotion™ PRO system.

Optionally said standard of care rehabilitative measure comprises GRASP.

Optionally said standard of care rehabilitative measure comprises at least one of reduced joint pain and improved motor function.

Optionally the method further comprises providing an improvement from baseline in upper extremity motor function measured by the Fugl-Meyer Assessment for Upper Extremity (FMA-UE) and/or its subscales as compared to said standard of care rehabilitative measure.

Optionally the method further comprises providing an improvement from baseline in upper extremity motor ability measured by the streamlined Wolf Motor Function Test (sWMFT) score as compared to said standard of care rehabilitative measure.

Optionally the method further comprises providing an improvement from baseline in self-care ability measured by the Barthel index (BI) as compared to said standard of care rehabilitative measure.

Optionally the method further comprises providing an improvement from baseline in functional independence measured by the Modified Ranking Scale (MRS) and/or associated disability-adjusted life year (DALY) as compared to said standard of care rehabilitative measure.

Optionally the method further comprises providing an improvement from baseline in the general health status as measured by the Stroke Impact scale (SIS) as compared to said standard of care rehabilitative measure.

Optionally the method further comprises providing an improvement from baseline in the severity of stroke symptoms as measured by the NIH stroke scale (NIHSS) as compared to said standard of care rehabilitative measure.

Optionally the method further comprises providing an improvement from baseline in arm function in daily activities as measured by the Motor Activity Log (MAL) as compared to said standard of care rehabilitative measure.

Optionally the method further comprises providing an improvement in motivation measured by the Intrinsic Motivation Index (IMI) as compared to said standard of care rehabilitative measure.

Optionally the method further comprises providing reduced therapist time spent administrating rehabilitation exercises as compared to said standard of care rehabilitative measure.

Optionally the method further comprises providing an improvement from baseline in upper extremity muscle strength measured by the Medical research Council Scale (MRC) as compared to said standard of care rehabilitative measure.

Optionally said muscle strength comprises one or more of strength for shoulder elevation, elbow flexion/extension, forearm pronation/supination and wrist extension/flexion.

Optionally said higher degree of therapeutic intensity comprises increasing an amount of time the patient spends during each therapeutic session, or increasing a number of exercises that the patient performs during said session, within a specific time frame, or both.

Optionally the method further comprises providing an increased rehabilitation dose as measured by the duration of the rehabilitation session without planned rest periods as compared to said standard of care rehabilitative measure.

Optionally the method further comprises performing the method during an acute period following a neurological trauma.

Optionally said neurological trauma comprises at least one of stroke and head injury.

Optionally the virtual object is displayed to the patient in an AR (augmented reality) or VR (virtual reality) environment.

Optionally the method further comprises determining a location of the virtual object in the AR or VR environment to avoid trunk involvement in a movement by the patient.

Optionally the method further comprises performing a calibration to determine a maximum reach of the patient and performing said determining said location according to a maximum of a range of 80-95% distance of said maximum reach of the patient.

Optionally said maximum distance is 95% of said maximum reach of the patient.

Optionally the method further comprises measuring an extent of trunk involvement according to movement of shoulders of the patient.

Optionally the method further comprises measuring EEG signals of the patient during said tracking said movement by the patient.

Optionally the method further comprises providing feedback to the patient according to said EEG signals.

Optionally the method further comprises providing feedback to the patient through a visual display of a mirror avatar.

Optionally the computational system comprises: a plurality of inertial sensors attached to the patient and an inertial sensor receiver for obtaining tracking data, a tracking engine for tracking the movements of the patient, and a data analysis layer for analyzing the tracked movements and for adjusting said displaying and said indicating according to said tracking.

According to at least some embodiments, there is provided a method for rehabilitating a patient with a computationally directed set of movements, the set of movements being directed by a computational system that comprises providing visual direction to the patient and tracking the movements of the patient, the method comprising: assessing an ability of the patient to perform a movement with a physical object; displaying a virtual object to the patient according to said assessing; indicating a movement to be performed by the patient with the virtual object, determined according to said assessing; tracking said movement; and adjusting said displaying and said indicating according to said tracking.

Optionally the computational system comprises: a depth camera and an RGB camera for obtaining tracking data, a tracking engine for tracking the movements of the patient, and a data analysis layer for analyzing the tracked movements and for adjusting said displaying and said indicating according to said tracking.

Optionally the virtual object is displayed to the patient in an AR (augmented reality) or VR (virtual reality) environment.

Optionally the computational system comprises a MindMotion™ PRO system.

Optionally the method further comprises receiving tracking data using an inertial sensor receiver of the computational system, the tracking data generated by a plurality of inertial sensors attached to the patient; analyzing data representing the tracked movements, using a data analysis layer of the computational system; and adjusting the displaying and the indicating according to the tracking using the data analysis layer of the computational system; wherein movements of the user are tracked using a tracking engine of the computational system.

According to at least some embodiments, there is provided a method for computationally directing a patient therapy, comprising: displaying a virtual object, using a display of the computational system; indicating a movement, using the display of the computational system, to be performed with the virtual object; tracking said movement, using a tracking sensor; receiving, using a data analysis layer, sensor data from the tracking sensor, the data analysis layer comprising a plurality of machine instructions; configuring one or more display instructions according to the tracking; and sending one or more display instructions to the display, the one or more display instructions to provide a visual direction on the display; wherein the indicating the movement and the configuring the one or more display instructions, indicating and tracking apply a higher degree of therapeutic intensity as compared to a standard of care rehabilitative measure.

Optionally the method further comprises assigning a visual indicator to the virtual object, the visual indicator communicating to the user to correct a trajectory.

According to at least some embodiments, there is provided a method with a computationally directed set of movements, the set of movements being directed by a computational system that comprises providing visual direction to the patient and tracking the movements of the patient, the computational system comprising a display, at least one tracking sensor and a plurality of machine instructions for controlling the display to provide the visual direction and for receiving sensor data from the tracking sensor to track the movements of the patient, the method comprising: displaying a virtual object; indicating a movement to be performed with the virtual object; tracking said movement; and adjusting said displaying and said indicating according to said tracking; wherein said displaying, indicating and tracking using a higher degree of therapeutic intensity as compared to a standard of care rehabilitative measure.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computer” on a “computer network,” it should be noted that optionally any device featuring a data processor and the ability to execute one or more instructions may be described as a computer or as a computational device, including but not limited to any type of personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, or a pager. Any two or more of such devices in communication with each other may optionally comprise a “computer network.”

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1A is a photograph that illustrates a user interacting with an exemplary system in accordance with embodiments of the invention. In this case, FIG. 1A illustrates a MindMotion™ PRO system of MindMaze SA.

FIG. 1B illustrates an exemplary system for providing rehabilitation with tracking in accordance with embodiments.

FIGS. 2A-2C illustrate exemplary displays of interactive rehabilitation exercises in accordance with embodiments. FIGS. 2A-2C are shown twice, once with color and once with reference numbers, for clarity.

FIG. 3 illustrates an exemplary diagram of a clinical trial in accordance with embodiments of the invention. In this case, FIG. 3 illustrates a diagram of an exemplary clinical trial entitled “Move-Rehab” while using a MindMotion™ PRO system.

FIG. 4A (right) and FIG. 4B (left) illustrate, for an exemplary embodiment, the location of virtual objects that can be used to induce the patient to perform specific motions that lead to an increase in ADL performance.

FIGS. 5A and 5B relate to an example of the placement of virtual objects for a particular set of movements in accordance with embodiments.

FIG. 6 relates to an exemplary schema for tests performed to validate the accuracy of systems in accordance with embodiments of the invention.

FIGS. 7A-7C illustrate an experimental configuration and results thereof for systems and methods in accordance with embodiments of the invention. In this case, FIGS. 7A-7C illustrate exemplary setup and results of a MindMotion™ PRO system;

FIGS. 8A and 8B illustrate an additional experimental configuration of a system in accordance with embodiments of the invention. In this case, a participant performing centered out movements over a 2D surface of a table in front of a MindMotion™ PRO system. A 3D camera tracks each participant's movements and projects on a feedback screen. Electroencephalogram can be acquired using an electrode cap and synchronized with exercise events measured through the 3D camera. (B) The approximate locations of the five virtual targets with respect to participant trunk location are described.

FIG. 8C illustrates an exemplary timeline of events in a reaching task that are valid for different conditions in accordance with various configurations.

FIG. 9A illustrates exemplary experimental conditions for healthy participants in accordance with embodiments. Direct Left, Direct Right and Mirror Right conditions are illustrated using real arm and corresponding VR-arm movements. At the bottom is illustrated the main comparison in studying VR-MVF.

FIG. 9B illustrates exemplary experimental conditions for a stroke participant in accordance with embodiments. Direct Left (Nonparetic), Mirror Left (Non-paretic) and Direct Right (Paretic) conditions are illustrated using real arm and VR-arm movements. At the bottom is illustrated the lesion side and main comparison in studying VR-MVF.

FIG. 10A shows grand-averages of MRCPs of healthy participants for Direct Left, Direct Right and Mirror Right conditions. The shaded area represents standard error of mean (S EM) computed separately for each condition at a given time point.

FIG. 10B shows topographic images of healthy participants' MRCPs at 800 ms for Direct Right, Mirror Right, Direct Left and difference between Mirror Right & Direct Right conditions.

FIG. 11A shows grand-averages of MRCPs of a stroke participant for Direct Left (Non-paretic) and Mirror Left (Non-paretic) conditions. The shaded area represents standard error of mean (S EM) computed separately for each condition at a given time point.

FIG. 11B shows topographic images of a stroke participant average MRCPs during the window [−225 −200] ms for Direct Left (Non-paretic) and Mirror Left (Nonparetic), and for the difference between Mirror Left (Non-paretic) and Direct Left (Non-paretic) conditions.

### DESCRIPTION OF AT LEAST SOME EMBODIMENTS

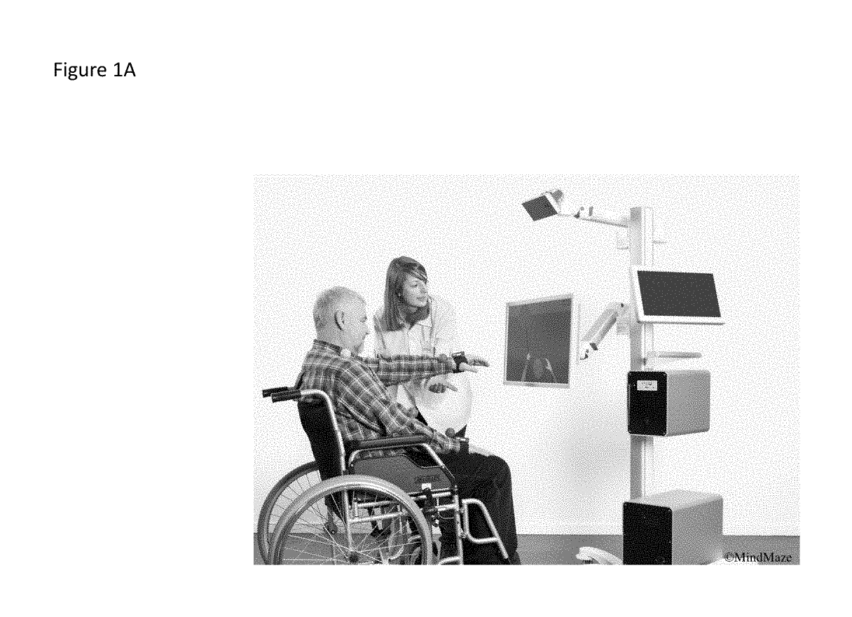

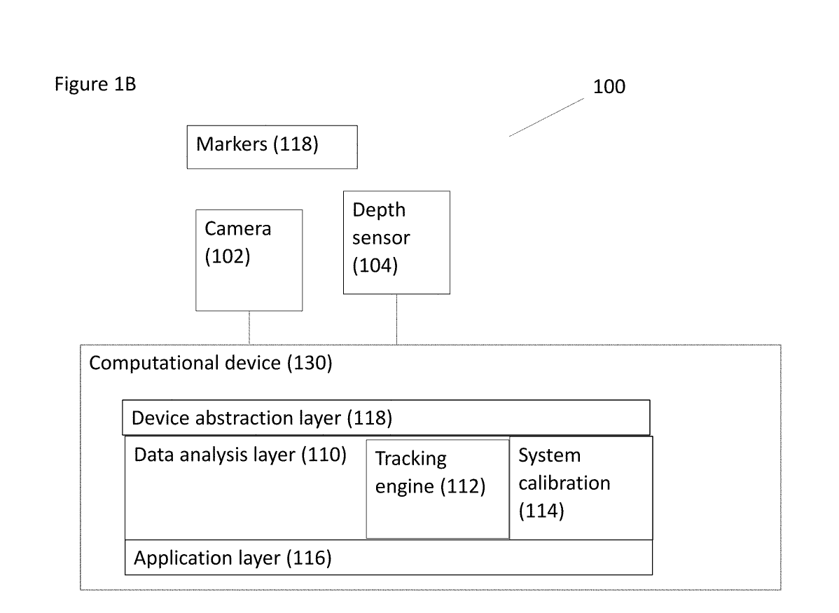

FIG. 1A shows a photograph that illustrates a user interacting with an exemplary system in accordance with embodiments of the invention. Here, the MindMotion™ PRO system of MindMaze SA is shown. FIG. 1B illustrates an exemplary system for providing rehabilitation with tracking, which incorporates a number of features of the MindMotion™ PRO system. Either or both systems may optionally be used with the methods as described herein.

Embodiments provides VR-based exercises for upper-limb neurorehabilitation after brain injuries. An implementation of the MindMotion™ PRO platform with immersive virtual reality (VR) is one exemplary embodiment. This platform is a mobile unit that includes a computing unit, a camera with stereo and depth sensors, and embedded 3D image processing system that captures motion by tracking the movement of six colored markers positioned on the joints, and two inertial hand sensors that add precision to the orientation of the subject's arm. Skilled artisans can appreciate that other embodiments need not be mobile, can include other or fewer components, and can include components configured in different ways. The MindMotion™ PRO platform is but one exemplary embodiment and should not be viewed as limiting to other embodiments. The colored markers are preferably active markers that emit a signal, such as LED lights for example, and more preferably emit different signals, such as different colored lights. Skilled artisans can appreciate that types of signals other than LEDs can be used. However, optionally no such markers are used in the form of devices attached to the subject, and the markers are instead data points that are detected to determine the location of particular joints in the data obtained, as described for example in PCT Application No. PCT/US18/17292, filed on Feb. 7, 2018, owned in common with the present application, which is hereby incorporated by reference as if fully set forth herein.

Optionally no inertial sensors are used; alternatively and optionally, the camera could be replaced by the inertial sensors. Optionally all markers are replaced by the inertial sensors.

Positions (3D cartesian coordinates) and orientations (quaternions) of the joints are mapped in real-time onto an avatar following the participant's movement. Motion data are recorded at a suitable sampling frequency, such as 30 Hz for example, and without limitation, and are stored in the computation unit for further analysis. Additionally, there are two screens for one for the exercises and one for the monitoring, respectively for the patient and the therapist, and a battery unit.

As described in greater detail below, optionally EEG signals (and/or optionally other biosignals) can be measured from the patient (not shown).

FIG. 1B shows a system 100 features a camera 102 and a depth sensor 104. Optionally camera 102 and depth sensor 104 are combined in a single product, such as the Kinect product of Microsoft, and/or as described with regard to U.S. Pat. No. 8,379,101, for example. Optionally, camera 102 and depth sensor 104 could be implemented with the LYRA camera of Mindmaze SA, for example, as implemented in a MindMotion™ PRO product. Preferably, camera 102 and depth sensor 104 are integrated which enables the orientation of camera 102 to be determined with respect to a canonical reference frame.

Sensor data preferably relates to the physical actions of a user (not shown), which are accessible to the sensors. For example, camera 102 may optionally collect video data of one or more movements of the user, while depth sensor 104 may provide data to determine the three-dimensional location of the user in space according to a distance of a part of the user from depth sensor 104. Depth sensor 104 preferably provides TOF (time of flight) data regarding the position of the user. The combination of sensor data from depth sensor 104 with video data from camera 102 allows a three-dimensional map of the user in the environment to be determined. As described in greater detail below, such a map enables the physical actions of the user to be accurately determined, for example, with regard to gestures made by the user.

To assist in the tracking process, optionally one or more markers 118 are placed on the body of a user. As used herein, the term “tracking” relates to tracking the movements of the user, whether through markers 118 or any other landmark or point(s) to follow. Markers 118 optionally feature a characteristic that can be detected by one or more of the sensors. Markers 118 are preferably detectable by camera 102, for example as optical markers. While such optical markers may be passive or active, preferably markers 118 are active optical markers, for example featuring an LED light. More preferably each of markers 118, or alternatively each pair of markers 118, comprises an LED light of a specific color which is then placed on a specific location of the body of the user. The different colors of the LED lights, placed at a specific location, convey a significant amount of information to the system through camera 102; as described in greater detail below, such information can be used to make the tracking process efficient and accurate. Alternatively, as described above, no such markers 118 are used and instead data points relating to specific joints are detected. Such markers, or the above described camera or other sensors for tracking the movements of the user, or a combination thereof may be described as a tracking device.

A computational device 130 can receive sensor data from camera 102 and depth sensor 104. Sensor data from other sensors can be received also. Any method steps performed herein may optionally be performed by such a computational device. Further, all modules and interfaces shown herein are assumed to incorporate, or to be operated by, a computational device, even if not shown. Optionally preprocessing is performed on the signal data from the sensors. Computational device 130 features a plurality of machine instructions controlling the display to provide the visual direction and for receiving sensor data from the tracking sensor to track the movements of the user.

Preprocessed signal data from the sensors can be passed to a data analysis layer 110, which preferably performs data analysis on the sensor data for consumption by an application layer 116. Application layer 116 can provide any type of interaction with a user. Preferably, such analysis includes tracking analysis, performed by a tracking engine 112. Tracking engine 112 preferably tracks the position of a user's body and preferably of one or more body parts of a user, including but not limited to one or more of arms, legs, hands, feet, head and so forth. Tracking engine 112 optionally decomposes data representing physical actions made by a user to data representing a series of gestures. A “gesture” in this case may optionally include an action taken by a plurality of body parts of a user, such as taking a step while swinging an arm, lifting an arm while bending forward, moving both arms and so forth. Such decomposition and gesture recognition could optionally be done separately.

Tracking data, generated by tracking engine 112, representing the tracking of a user's body and/or body parts, optionally decomposed to data representing a series of gestures, can then be received by application layer 116, which can translate the data representing physical actions of a user into data representing some type of reaction, analyzes this reaction data to determine one or more action parameters, or both. For example, and without limitation, a physical action taken by the user to lift an arm is a gesture and is captured by cameras and sensors (102, 104) among other possible sensors which generate sensor data. The sensor data could be translated by application layer 116 to data for generating an image of an arm lifting a virtual object. Alternatively, or additionally, such data representing a physical action could be analyzed by application layer 116 to determine the user's range of motion or ability to perform the action. Application layer 116 could for example provide a game for the user to perform as described herein.

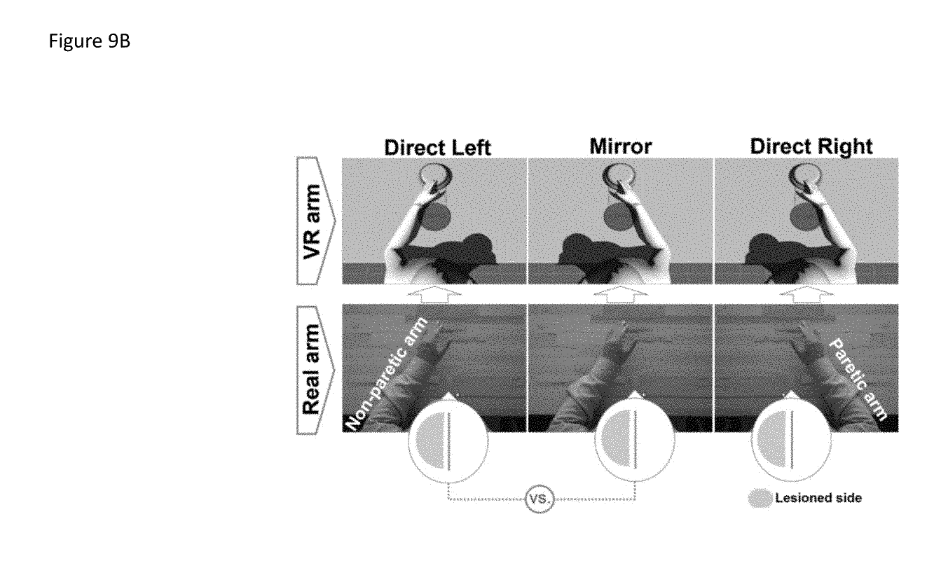

Optionally, application layer 116 could create a mirror avatar to provide feedback, which would mirror the user's motions and provide a visual display of such motions. Such a mirror avatar is illustrated and described below in connection with FIGS. 9A and 9B.

Data analysis layer 110 also preferably includes a system calibration module 114. As described in greater detail below, system calibration module 114 can calibrate the system in regard to the position of the user, in order for the system to be able to track the user effectively. System calibration module 114 may optionally perform calibration of the camera 102, depth sensor 104, as well as any other sensors, in regard to the requirements of the operation of application layer 116; however, preferably device abstraction layer 108 performs any sensor specific calibration. Optionally, the sensors may be packaged in a device, such as the Kinect, which performs its own sensor specific calibration.

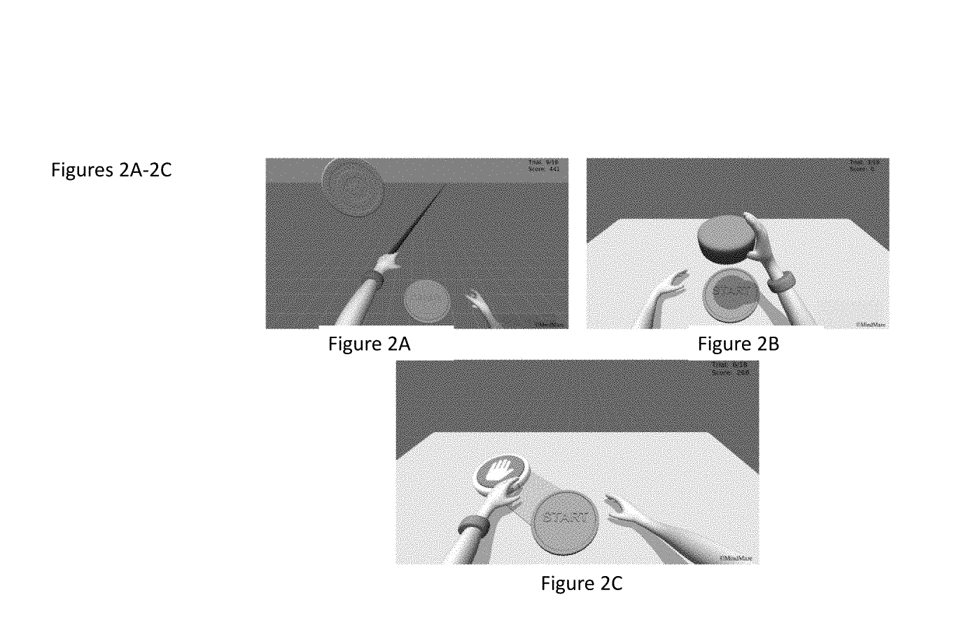

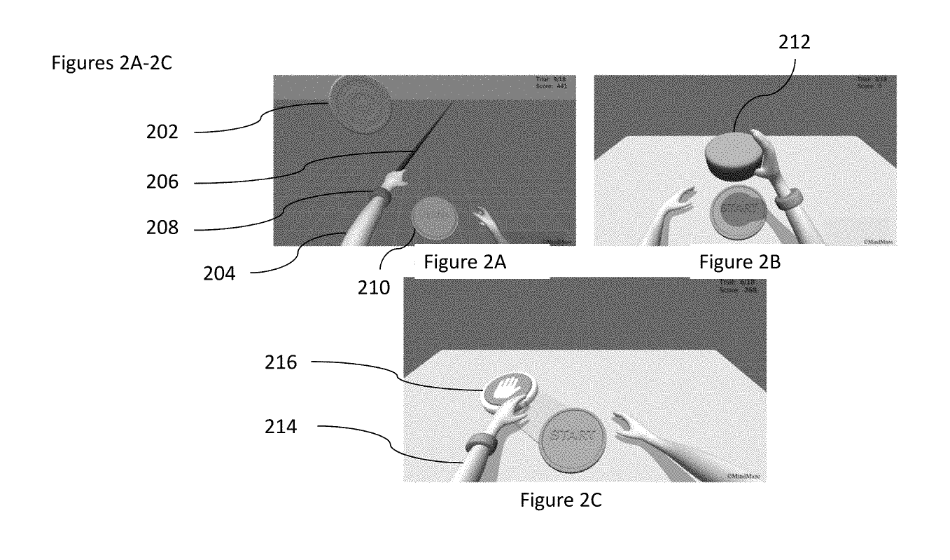

FIGS. 2A-2C show exemplary screenshots that can be generated by application layer 116 in embodiments. In this case, the screenshots of FIGS. 2A-2C are examples from a MindMotion™ PRO system when displaying different interactive rehabilitation games. For this particular embodiment, a game begins, when a start button, or start-pad, appears in the virtual environment, inviting the participant to position his hand on it. Once it is done, a target appears and the participant can start the exercise. In this particular embodiment, the rehabilitation system is composed of four exercises: point, reaching, grasp and fruits champion. Skilled artisans can appreciate that other exercises can be provided by different embodiments. Referring to the pointing exercise of the exemplary embodiment illustrated in FIG. 2A, the exercise includes aiming to the center of a target 202 with the arm 204 and stay for a few seconds. Referring to the grasping exercise of the exemplary embodiment illustrated in FIG. 2B, the exercise includes catching an object 212 in the vertical plane and dropping it in a new location. Referring to the reaching exercise of this exemplary embodiment illustrated in FIG. 2C, a user extends an arm 214 to hit a target 216. Finally, the fruit champion exercise, (not shown), includes displaying virtual fruits for a user to virtually cut. Targets (202, 216) and objects (212) can be located in predefined positions that depend on the difficulty level. In preferred embodiments, the positions are generated randomly and distributed equally across the number of repetitions of an exercise.

In preferred embodiments, after each repetition of an exercise, a score appears informing the player about his performance during the task. Additionally, in some preferred embodiments, if a task is not completed within 5 s, a timeout warning appears and the exercise resumes. For example, in the reaching exercise of the embodiment illustrated in FIG. 2A, the path 206 and wrist-band 208 are blue after the start-pad 210 was hit to indicate the exercise has started. Then, if the followed trajectory respects the specified path 206, the path 206 and wrist-band 208 turn green to indicate the exercise was successful. On the other hand, divergence from the path 206 provoke a change of color to red, inviting the user to correct the trajectory of the arm 204. Skilled artisans can appreciate that other indicators and methods of indicating commencement, conclusion, success, failure, and the like of exercises can be used.

Game events, or triggers, can be determined by the super-position of collision volumes of the hand and the elements of the game (for example, start-pad 210, path 206, target 202, and the like). Events can include: contact with the start-pad, contact with the target, mistake feedback on, success feedback on, timeout feedback on, and the like. The beginning of movement can be defined as when the collision volumes of the hand and start-pad do not superimpose anymore, and the end of movement is set when the tip of the participant's fingers reach the target.

The systems and games as described herein may be implemented in an overall plan for rehabilitation, and are currently being tested in a clinical trial, as described with regard to Example 1 below.

An additional plan for implementing such systems and games for rehabilitation that leads to an increase in an ability to perform ADL (activities of daily living) is described with regard to Example 2 below.

### Example 1—MindMotion™ PRO in “Move-Rehab” Clinical Trial

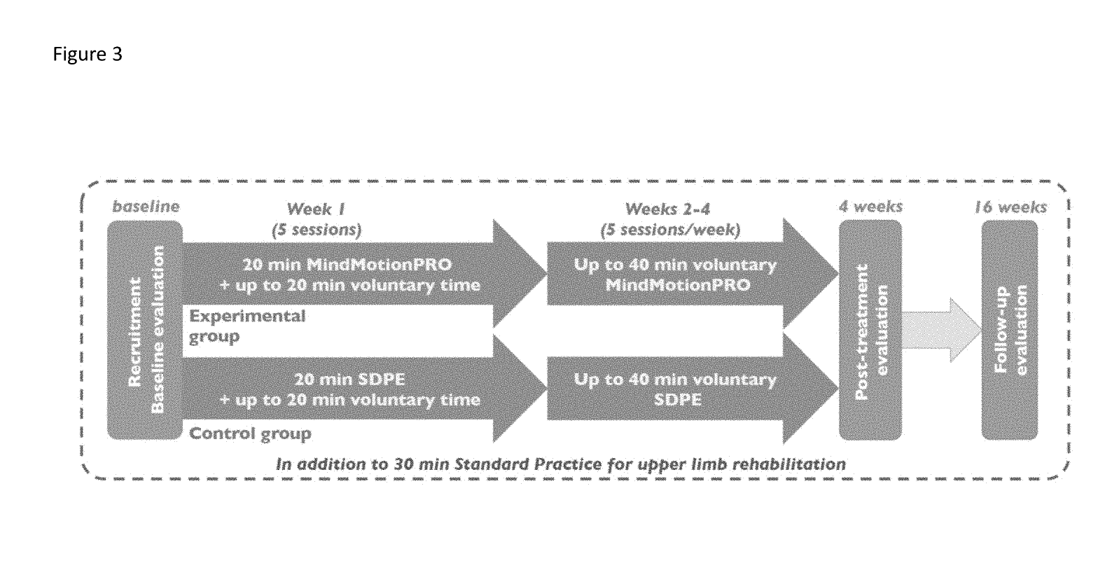

FIG. 3 relates to a diagram of a clinical trial entitled “Move-Rehab”, currently underway, that is using the MindMotion™ PRO system.

The Move-Rehab clinical trial protocol may be found at https://clinicaltrials.gov/ct2/show/NCT02688413, and is described below briefly for clarity only. The trial is intended to show that exercises according to methods which embody the invention or which use systems in accordance with embodiments of the invention, including exercises embodied in MindMotion™ PRO exercises, can deliver a higher degree of therapeutic intensity as compared to standard of care rehabilitative measures that are currently considered to be intensive. The standard of care protocol that is being used for the comparison is called GRASP (Graded Repetitive Arm Supplementary Program). GRASP is an arm and hand exercise program for stroke patients. Therapeutic intensity is a combination of the amount of time that a patient can spend during each therapeutic session, plus the number of exercises that the patient can do during each session, within a specific time frame. Two ways to increase therapeutic intensity are by increasing the amount of time spent or by increasing the number of exercises performed during a particular period of time.

In addition, the clinical trial is intended to determine whether the standard of care in accordance with embodiments of the invention, including the standard of care delivered by the MindMotion™ PRO platform, is more effective than the standard of care, which is again GRASP, in comparison to the below list of changes from baseline in comparison to the standard of care.

Further discussion of the clinical trial protocol reference MindMotion™ PRO. It is to be understood that such references are but one preferred embodiment and do not operate to limit the invention.

Purpose

Randomized controlled multi-centered study using MindMotion™ PRO, an immersive virtual reality-based system for upper limb motor rehabilitation in early post-stroke patients. The study aims to evaluate the ability of MindMotion™ PRO technology to increase the rehabilitation dose. Effectiveness will be evaluated by validated rehabilitation performance scales. Cost-effectiveness will be assessed by the resource utilization. Table 1 shows a summary.

TABLE 1
Study Summary

| Condition | Intervention |
|---|---|
| Stroke | Device: MindMotion ™ PRO |
| Motor Disorders | Other: Self-Directed Prescribed Exercises |

Study Type: Interventional
Study Design: Allocation: Randomized
   Intervention Model: Parallel Assignment
   Masking: Single Blind (Outcomes Assessor)
   Primary Purpose: Treatment
Official Title: Randomized Parallel-group Study Evaluating the Effectiveness and Cost-effectiveness of the Co-administration of MindMotion™ PRO Plus Standard Practice Versus Standard Practice in Early Post-stroke Upper-limb Rehabilitation

### Further Study Details:

### Primary Outcome Measures:

Rehabilitation dose as measured by the duration of the rehabilitation session without planned rest periods [Time Frame: 4 weeks]

### Secondary Outcome Measures:

Number of exercises performed [Time Frame: 4 weeks]
Change from Baseline in upper extremity motor function measured by the Fugl-Meyer Assessment for Upper Extremity (FMA-UE) and its subscales [Time Frame: baseline, 4 weeks, 16 weeks]
Change from Baseline in upper extremity motor ability measured by the streamlined Wolf Motor Function Test (sWMFT) score [Time Frame: baseline, 4 weeks, 16 weeks]
Change from Baseline in self-care ability measured by the Barthel index (BI) [Time Frame: baseline, 4 weeks, 16 weeks]
Change from Baseline in functional independence measured by the Modified Ranking Scale (MRS) and associated disability-adjusted life year (DALY) [Time Frame: baseline, 4 weeks, 16 weeks]
Change from Baseline in the general health status as measured by the Stroke Impact scale (SIS) [Time Frame: baseline, 4 weeks, 16 weeks]
Change from Baseline in the severity of stroke symptoms as measured by the NIH stroke scale (NIHSS) [Time Frame: baseline, 4 weeks, 16 weeks]
Change from Baseline in arm function in daily activities as measured by the Motor Activity Log (MAL) [Time Frame: baseline, 4 weeks, 16 weeks]
Motivation measured by the Intrinsic Motivation Index (IMI) [Time Frame: 1 week and 4 weeks]

### Other Outcome Measures:

Resource utilization: time spent administrating rehabilitation exercises [Time Frame: 4 weeks] therapist (physiotherapist or other medical staff) time spent administrating rehabilitation exercises
Change from Baseline in upper extremity muscle strength measured by the Medical research Council Scale (MRC) [Time Frame: Baseline, 1 week, 2 weeks, 3 weeks, 4 weeks and 16 weeks] muscle strength for shoulder elevation, elbow flexion/extension, forearm pronation/supination and wrist extension/flexion.

Table 2 shows an effect on the arms.

TABLE 2
Summary of the Arms

| Arms | Assigned Interventions |
|---|---|
| Experimental: | Device: MindMotion ™ PRO |
| MindMotion ™ PRO | The MindMotion ™ PRO, a certified device for medical use, is a |
| MindMotion ™ PRO | virtual reality based system to train upper limb activities in a |
| exercises in addition to | game scenario. The participant will receive 5 exercises sessions |
| standard practice for upper | with the MindMotion ™ PRO per week over 4 weeks. This is |
| limb rehabilitation | done in addition to standard practice for upper limb |
| | rehabilitation which should be at least 30 min five times per |
| | week. |
| Active Comparator: Self- | Other: Self-Directed Prescribed Exercises |
| Directed Prescribed | GRASP is an arm and hand exercise program for stroke |
| Exercises | patients, designed to supplement standard rehabilitation |
| Self-Directed Prescribed | therapies. The participant will receive 5 GRASP exercises |
| Exercises in addition to | sessions per week over 4 weeks. This is done in addition to |
| standard practice for upper | standard practice for upper limb rehabilitation which should be |
| limb rehabilitation | at least 30 min five times per week. |
| | Other Name: Graded Repetitive Arm Supplementary Program |
| | (GRASP) |

### DETAILED DESCRIPTION

The goal of the study is to show that the MindMotion™ PRO platform (or other embodiments) is a tool that allows a patient to increase the amount of rehabilitation therapy performed. The study measures the rehabilitation dose, as measured by the duration of the rehabilitation session and the number of exercises performed by the patient. The study hypothesis is that patients in the MindMotion™ PRO group spend more time performing rehabilitation exercises than in the Self-Directed Prescribed exercises group and, thus, receive more effective rehabilitation. The effectiveness of the MindMotion™ PRO versus Self-Directed Prescribed Exercises are also measured, based on the change in rehabilitation performance measures. The cost-effectiveness is measured by the resource utilization, as defined by the time spent by the therapist providing the rehabilitation session.

### Eligibility

Ages Eligible for Study: 18 Years and older (Adult, Senior)

### Sexes Eligible for Study: All

### Accepts Healthy Volunteers: No

### Criteria

### Inclusion Criteria:

Male/female >18 years old
First ever unilateral supratentorial ischemic stroke with contralateral upper extremity weakness
1 to 6 weeks post-stroke
Able to give informed consent
Not participating any other intervention studies
Experiencing motor difficulties in using the paretic arm, with a FMA-UE score in the range of 20 to 40 out of 66
Stroke severity with NIHSS score between 5 (mild) and 14 (moderate) out of 42
The participant is expected to remain available (geographically stable) for 4 months after enrolment.

### Exclusion Criteria:

Any medical condition compromising the safety or the ability to take part to the study (such as insufficient vision or hearing, inability to participate to therapy session, inability to communicate, upper limb condition not linked to stroke, uncontrolled blood pressure, uncontrolled diabetes, co-morbidity)
Recurrent and moderate to high upper limb pain limiting delivery of rehabilitation dose
History of more than one epileptic seizures since stroke onset or uncontrolled epileptic seizure
Mild to severe cognitive impairment (Mini mental state exam (MMSE) score <24/30)
Depression (Hospital Anxiety and Depression Scale >8/21)
Moderate to severe hemispatial neglect compromising the ability to take part to the study, as determined by the Bells tests (>6 errors)
Brain stem stroke

### Example 2—MindMotion™ PRO for Rehabilitation to Improve ADL

It is highly desirable to improve ADL for rehabilitation of patients, particularly for stroke victims. As noted in the Background, rehabilitation for such patients can be quite difficult, due to lack of necessary therapists and therapist resources to fully impact all patients.

The method described herein, which is described as being implemented with MindMotion™ PRO but which may, in fact, be implemented with other systems and methods in accordance with embodiments of the invention as described herein, relates to a specific rehabilitation process that leads to an increase in the ability of the patient to perform ADL.

Part of the consideration relates to outcomes of specific motions that could lead to an improvement in ADL performance by the patient. These outcomes are listed below.

Tables 3 and 4 list desired outcomes that could be measured and improved by a system as described herein, upon interacting with the patient. RoM as used herein relates to range of motion.

TABLE 3

| OUTCOME | ASSESSMENT | |
|---|---|---|
| NO. | PARAMETERS | HOW TO GET IT |
| 1 | Elbow RoM | Far away reach |
| 2 | Smoothness | Long straight-line trajectory |
| 3 | Trunk displacement and | Far away reach |
| | rotation | |
| 4 | Shoulder Elevation | Reaching on high platforms |

TABLE 4

| OUTCOME | | |
|---|---|---|
| NO. | OTHER PARAMETERS | HOW TO GET IT |
| 5 | Realistic movement with | Use real objects |
| | object manipulation | |
| 6 | Easy movements for | Closer objects, gross motor |
| | severely impaired | skills |
| 7 | Difficult movements for | Fine motor skills, father |
| | mildly impaired | distances, smaller objects, faster |

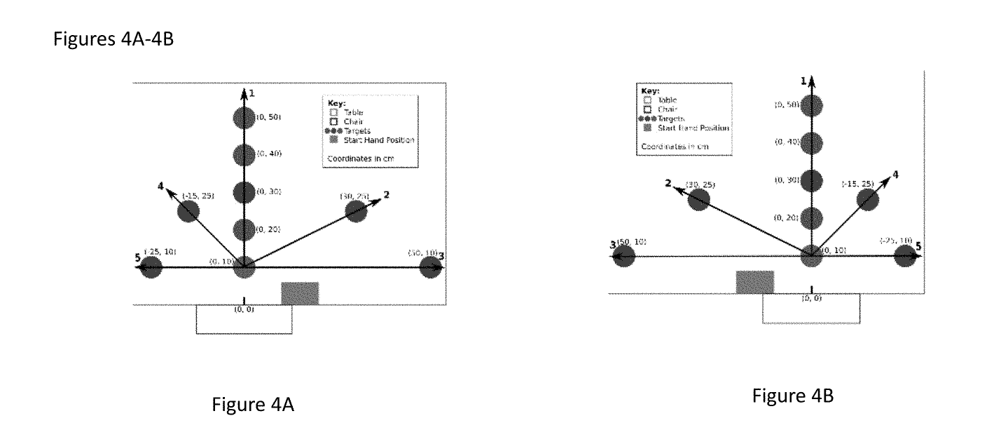

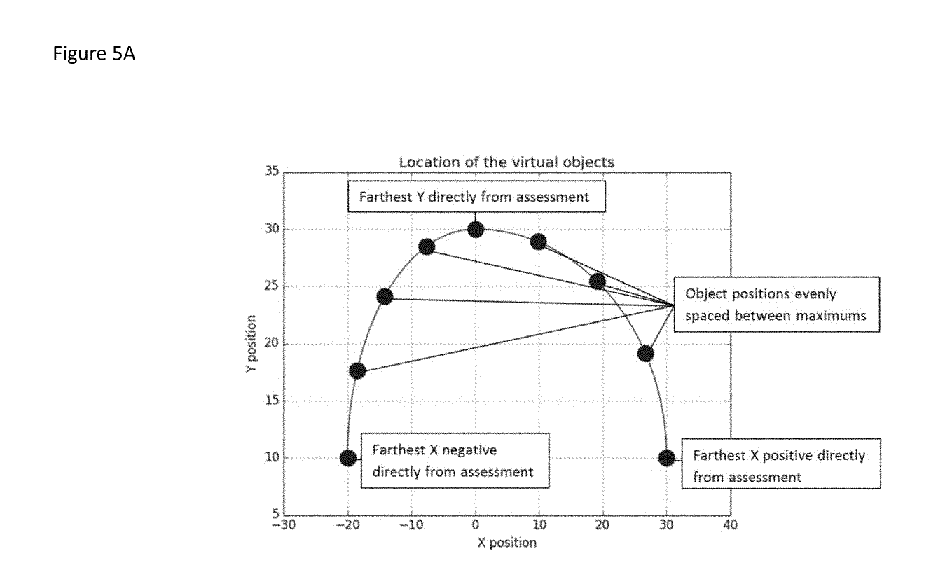

FIG. 4A and FIG. 4B illustrate, for an exemplary embodiment, the location of virtual objects that can be used to induce the patient to perform specific motions that lead to an increase in ADL performance. The term “virtual objects” relates to any virtual object displayed as part of rehabilitation activity or game described herein. The diagram in FIG. 4A shows the location of such objects that may be manipulated by the right hand, while the diagram in FIG. 4B shows the location of such objects that may be manipulated by the left hand. In addition, the diagrams of FIGS. 4A and 4B also show the placement of actual physical objects for manipulation by the patient, for example to test whether the virtual tasks by MindMotion™ PRO invoke the same or similar movements as equivalent tasks with physical objects, and/or whether such virtual tasks invoke the same intensity of movement. Furthermore, equivalency testing with physical objects placed in that manner may also be used to show whether the patient has improved capabilities after performing one or more sessions with virtual objects.

For each activity, such as a game, the start hand position=hand placed on the table so that the wrist joint is aligned with the table edge. the arm should be positioned with elbow flexion: 90°, shoulder flexion: 0°, shoulder abduction: 0°.

The game would induce the patient to reach for a virtual object at the positions shown which would lead to an improvement in the patient's ability to perform ADL.

Defining the Workspace:

In the acute phase, for example following a neurological trauma (including but not limited to a stroke or traumatic head injury), it is desired to avoid compensation as the brain is highly plastic so there is a better chance of true recovery. The workspace is defined below with regard to the goals to be reached.

X-Y direction: reach exercise should be the maximal arm extension without compensation. See if in grab exercise there is trunk compensation, if yes find the distance at which it starts. Find distance at which compensation starts in place exercise.

Use the maximal displacement out of the first 3 exercises of Table 3 to define the maximal Y distance. Use the maximal and minimal X values from the 1 reach exercise.

Z direction: using exercises 5-7 of Table 3 find maximal height reached.

### Table 5—MindMotion™ PRO System Exercise Descriptions

Specific examples of such games may optionally be performed by interactions with the MindMotion™ PRO system or, again, other embodiments, as described in greater detail below, in Table 5. All exercises may be set with a 25 s time-out for example.

TABLE 5
a - MINDMAZE PRO EXERCISE DESCRIPTIONS

| EXERCISE/GAME | SKILL | DESCRIPTION |
|---|---|---|
| Point | Forearm stability to develop | Point towards a virtual target |
| | shoulder and elbow strength, | (in the “air”) by the moving |
| | RoM, and control | forearm |
| | | Keep pointed for 2 s |
| Point-Hand | Hand and forearm stability to | Point towards a target by |
| | develop shoulder and elbow | moving the forearm and/or by |
| | strength, RoM, and control | moving the wrist |
| | | Keep pointed for 2 s |
| Reach | Arm (or hand) | To reach for a virtual target |
| (-Hand) | Trajectory to develop | placed on the table and stay |
| | shoulder and elbow strength, | 0.5 s on the target. |
| | RoM, and control | |
| Grasp | Two-step arm (or hand) | To reach for a virtual object |
| (-Hand) | trajectory to develop | placed above start pad and to |
| | shoulder, elbow, forearm, and | place it on a virtual target for |
| | wrist strength, RoM, and | 1 s at table level |
| | control | |
| Fruit Champion | Aimed at developing | The exercise/game requires |
| | shoulder, elbow, forearm and | the patient/player to slice |
| | wrist strength, RoM, and | through virtual fruits that |
| | motion control while | appear on the display at |
| | increasing speed and | various 3D positions using a |
| | accuracy | virtual katana while avoiding |
| | | cutting virtual rocks |

b - MINDMAZE PRO EXERCISE DESCRIPTIONS

| | | WHAT CAN BE | |
|---|---|---|---|
| EXERCISE/GAME | CURRENT OBJECT | VARIED | PARAMETERS |
| Point | Level 1: 3 ø90 cm | Target size, position | Wrist RoM |
| | targets at 200 cm from | and height | Smaller targets |
| | start-pad, and 10 cm | Time before timeout | and timeout with |
| | high. | number of repetitions | better scores |
| Point-Hand | Level 2: 5 ø60 cm | | |
| | targets at 200 cm from | | |
| | start-pad and 10 cm | | |
| | high. | | |
| | Level 3: 7 ø48 cm | | |
| | targets at 200 cm from | | |
| | the start-pad either | | |
| | 10 cm or 40 cm high. | | |
| Reach | Level 1: 3 ø18 cm | Target size and | Height platform |
| (-Hand) | targets 24 cm away | position Time before | sequence |
| | from the startpad | timeout Could use | performance for |
| | Level 2: 5 ø16 cm | different heights for | object height |
| | targets 35 cm away | the targets number of | Smaller targets |
| | from the start-pad | repetitions | and timeout with |
| | Level 3: 7 ø12 cm | | better scores |
| | targets at 37.5 cm from | | |
| | the start-pad | | |
| Grasp | Targets same | Object height and | Height platform |
| (-Hand) | placement as reach | position | sequence |
| | exercise but different | Target height and | Performance for |
| | sizes. | position and size | object height |
| | Level 1, 2: ø22 cm, | Time before timeout | Smaller targets |
| | Level 3: ø19 cm | number of repetitions | and timeout with |
| | The Object is always | | better scores |
| | placed 25 cm above the | | |
| | start-pad | | |
| Fruit Champion | Not mentioned | Fruit and rock size, | Speed and time of |
| | | position and whether | movement |
| | | they are static or not | |
| | | Speed of moving | |
| | | objects | |
| | | Number of fruits at | |
| | | the same time | |
| | | Display time of fruit | |
| | | Number of fruit to cut | |
| | | in one session | |

To test the above games for their ability to improve ADL performance, a specific set of movements were selected as described with regard to Table 6.

TABLE 6
MOST POPULAR PARAMETERS USED TO MEASURE UPPER
LIMB KINEMATICS OF STROKE SURVIVORS

| Movement parameter | Movement to put into evidence |
|---|---|
| Smoothness (NMUs) | Long straight line trajectory |
| Elbow ROM | Far away reach |
| Trunk displacement and rotation | Far away |
| reach | |
| Shoulder elevation | Reaching on high platforms |
| Duration | Any |

| | MOVEMENT TO PUT INTO |
|---|---|
| MOVEMENT PARAMETER | EVIDENCE |
| Smoothness (NMUs) | Long straight-line trajectory |
| Elbow ROM | Far away reach |
| Trunk displacement and rotation reach | Far away |
| Shoulder elevation | Reaching on high platforms |
| Duration | Any |

A long straight-line trajectory would emphasize the smoothness parameter as the longer the trajectory, the higher the possible NMUs. A far reach would cause high elbow ROM or/and trunk displacement. A mid-sagittal plane reach would avoid having to distinguish between natural trunk rotation and compensatory trunk rotation. Shoulder elevation can be emphasized by reaching for objects on high platforms.

The first sequence of movements was chosen to find the maximal elbow flexion. The subjects were therefore asked to reach as far away as possible by sliding their hand along the table in five different directions while keeping their back against the chair. In order to then find the maximal possible reach, and to find at what point trunk compensation starts, the second movement chosen was to take a cup and place it at different distances on the table along the mid-sagittal plane. These movements belong to the function domain of the ICF. The remaining movement sequences were based on activities of daily living.

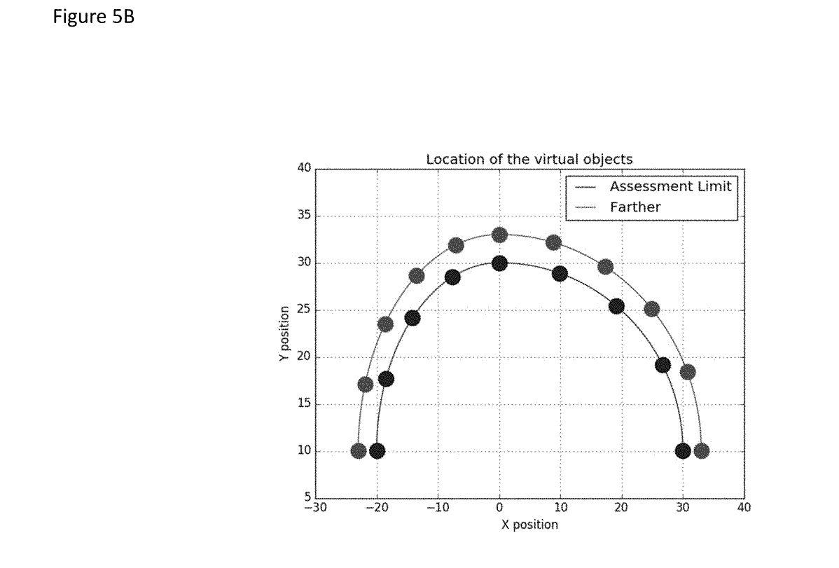

FIGS. 5A and 5B show how a series of movements could be determined for a particular exercise and then performed, in terms of virtual object placement. Optionally the ability of the subject to reach for, grasp and move objects is first determined according to FIGS. 4A and 4B, whether with actual physical objects, virtual objects or both.

An assessment of the movement capabilities of the patient is first performed, for example as described above. From such an assessment, the farthest distance in the Y (away from the patient, Ymax) and X (to the side of the patient, Xmax and Xmin) directions can be decided, for object placement. The placement of the objects between will be placed along the ellipse made by the maximal positions according to the following equation:

$${( \frac{x}{a} )}^{2} + {( \frac{y}{b} )}^{2} = 1 ( a \text{:} major axis ( X max and X min in this case ) , b \text{:} minor axis ( Y max ) ) .$$

FIG. 5A shows an example of a patient who would have reached with his or her right hand 30 cm away from the start pad, 30 cm to the right of the start pad, and 20 cm to the left.

During game time, a certain percentage of the movements would be made to reach objects at the assessment limit, set as x %, and (100-x) % farther than the limit, such as for example 80% at the limit and 20% beyond the limit. If there was a sequence of 10 movements for one exercise for example, 8 of the movements would be to the limit points (blue in FIG. 5B) and 2 would be to the points farther away (green in FIG. 5B). This object placement would induce the patient to continually improve, while reducing frustration and increasing the potential therapeutic time period.

One popular specific task for rehabilitation for ADL is a drinking task which comprises reaching, grasping, transporting, drinking, and placing a water filled cup. This task uses a cup filled with water, therefore an adapted version of this task avoiding spillage was used. The subjects were asked to pretend to drink from an empty weighted cup. This task may be impossible for severely impaired patients, and therefore a more general task, which includes reaching, grasping, transporting, and placing a cup was also chosen. A few other references, as well as the ones for the drinking task, also include these aspects. Many ADLs include this series of aspects such as drinking, eating, teeth brushing, shampooing etc. and mostly include bringing an object towards oneself. For this reason, a task to grab a cup and bring it towards oneself was chosen. This task was adapted to have easy and difficult versions by having the objects placed in different positions (contralateral, ipsilateral and midsagittal) and at four different heights.

According to Fitts' law (described in P. M. Fitts, “The information capacity of the human motor system in controlling the amplitude of movement,” Journal of Experimental Psychology, vol. 47, pp. 381-391, June 1954), the farther the movement distance, the shorter the time, and the higher the precision, the more difficult a movement is. Therefore, reaching for a small object far away is more difficult than reaching for a large object that is close. Even though the MindMotion™ PRO system as used in the experiment does not track finger movements, a more difficult fine motor skill task was included as a movement sequence to see if the proximal joints perform differently than with easier tasks. The chosen task was to take a spoon and put it in the cup, and then take a sugar cube to put in the cup. The last part of the sequence includes bringing the cup back towards oneself to see if this movement is performed at a slower pace than earlier. This was included to verify for fatigue.

The task of grasping a real cup, as an ADL, was tracked with the MindMotion™ PRO technology, to determine the motor performance. This information is being used to adapt the MindMotion™ PRO game activities to specifically connect the missing or reduced capabilities to the games that the patient performs. Based on the connection between the patient's deficits in an ADL activity and the MindMotion™ PRO, the MindMotion™ PRO activities are adapted for the specific patient, to overcome these deficits.

The following concepts are tested: correlation between the performance of a healthy subject in an ADL and the motions that can be decomposed to MindMotion™ PRO activities. Further, it is validated that the MindMotion™ PRO activities actually demand the same movements that need to be improved.

### Example 3—Validation of Accuracy of MindMotion™ PRO

The accuracy of the MindMotion™ PRO was validated through experimental testing, which demonstrated that the MindMotion™ PRO can track and measure movements with a high degree of accuracy.

Methods

A draw-wire encoder (Phidgets encoder ENC4104_0) with a 0.08 mm displacement resolution was used. A joining piece was 3D-printed to couple the marker to the encoder, and another one to screw to the encoder to be able to clamp the device to the table. Various tests were then performed, in terms of various movements that were performed:

1) at a natural pace and at the fastest pace the tester could move.

2) parallel to the camera close, far, and central on the testing table and perpendicular to the camera right, left and central on the testing table

3) with a camera tilted downwards at two different angles

4) along the axes of the left arm movement assessment schema, as this represents the region of the movements subjects perform during the assessment.

5) with a new and old camera calibration

The linearity of the movement was guaranteed by clamping a plastic board to the table and sliding the maker/cable joining piece along the edge of it. For each test the movement was repeated five or ten times in each direction.

The analysis of the data included finding the difference of the maximal measured displacement of the two systems, and the percentage error as followed:

$${Disp}_{diff} [ mm ] = {max Disp}_{marker} - {max Disp}_{encoder} error [ \% ] = \frac{100 \cdot {Disp}_{diff}}{{max}_{encoder}}$$

Therefore, a positive difference indicates an overestimation of the maximal displacement and a negative difference an underestimation of the maximal displacement. Moreover, the Bland-Altman graph of each movement was plotted (see J. M. Bland and D. Altman, “Statistical methods for assessing agreement between two methods of clinical measurement,” The Lancet, vol. 327, no. 8476, pp. 307-310, 1986, available at: http://dx.doi.org/10.1016/S0140-6736(86)90837-8). This corresponds to plotting the average of both displacement values against the difference between them at each time point.

In order to plot these, the movement graphs (x-axis: time, y-axis: displacement) had to be temporally aligned. This was done by finding the moment when the displacement value was 50% of the averaged maximal displacement, and aligning the movement plots at this point. Moreover the temporal rate had to be the same in order to compare the displacement of the two systems at each time point. Since the marker data was recorded with a rate of 30 Hz, and the encoder recorded data did not have a steady rate (as it only recorded data at a change of displacement, maximum 140 Hz for fast movements), the encoder data was interpolated linearly and replotted with the same time points as the marker data.

A static validation was also implemented. This validation included filming the six markers attached to a board at specific distances from each other and verifying that the measured distance between markers remain the same no matter the recording day, the marker board position and orientation, and the camera orientation. The six markers were attached in the same disposition as they would be on a patient, that is the two shoulder markers aligned, the two elbow markers below those and then wrist markers below again, and taking into account left and right. Moreover, a static validation for the measurement of height was performed. A marker was placed on the table and on platforms of different heights used for the upper limb assessment. The platforms were 3D printed at heights of 11, 23 and 38 cm. The platforms with the markers were placed on the same table position one after the other. They were then filmed on different days, to verify that the heights measured by the camera system remain the same.

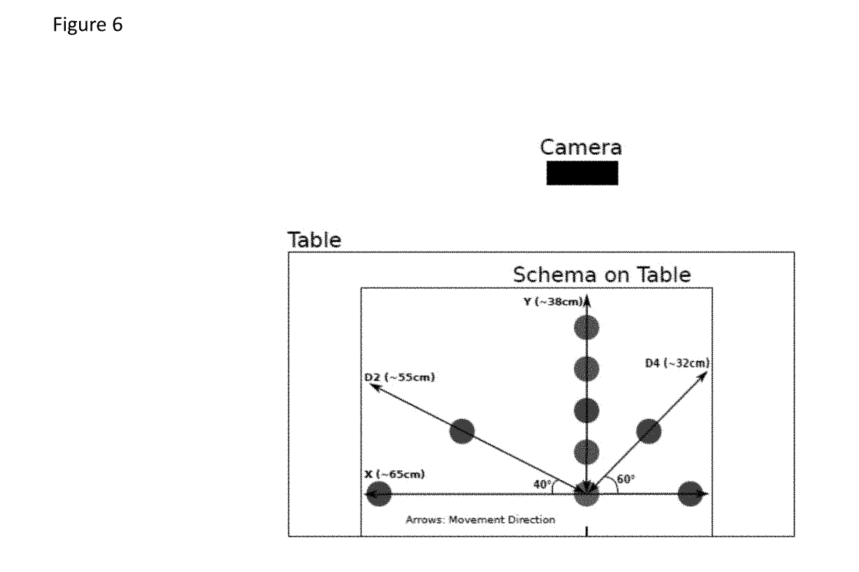

FIG. 6 shows a schematic diagram showing the movement direction of the marker/wire coupling on the left arm movement assessment schema (previously described with regard to FIG. 4B). For all test results, data is summarized but not shown.

The first test was performed with regard to speed of movement: Each movement along the arrows of the assessment schema (X, Y, D2, D4 in FIG. 6A) was performed five times on two different dates (indicated by a and b in the plot). The difference between the displacement error of a movement completed at natural pace and fastest possible pace was negligible.

Location of movement in the camera's field of view: Each movement was performed five times for each camera orientation. Again, the errors were very low or negligible.

With the camera tilted downwards at two different angles: changing the downward orientation of the camera had little or no effect.

With a new and old camera calibration: The movements were processed once with the calibrated camera data from 13 months previous to the test, and with the calibrated camera data from a new calibration. Similar data was obtained on both occasions, indicating the stability of the calibration.

Along the arrows of the assessment scale: For the displacement validation performed along the arrows of the assessment scale, each movement was plotted on a displacement against time graph to verify that the temporal alignment was correct. Overall, the results show high repeatability.

Static validation: The static validation to verify the displacement between the markers was performed on multiple occasions. The deviation between measurements was very low or negligible.

### Example 4—Performance of MindMotion™ PRO with Healthy Subjects

The performance of the MindMotion™ PRO system was tested with 10 healthy subjects. The tests demonstrated that MindMotion™ PRO could accurately measure the movements of the subjects. In addition, various adjustments to the games and system operation were also determined and are described herein. An exemplary set of measurements for determining trunk displacement with the MindMotion™ PRO system is also described.

Methods

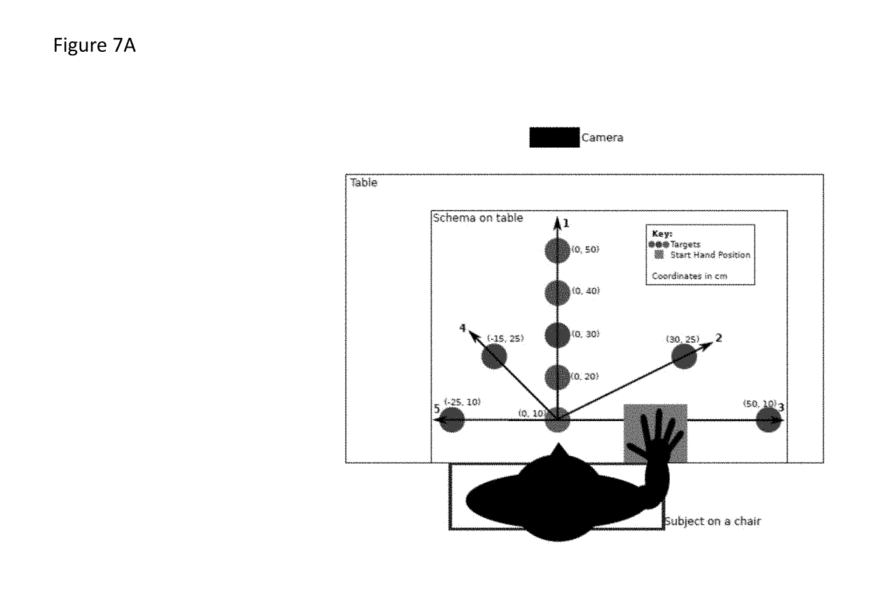

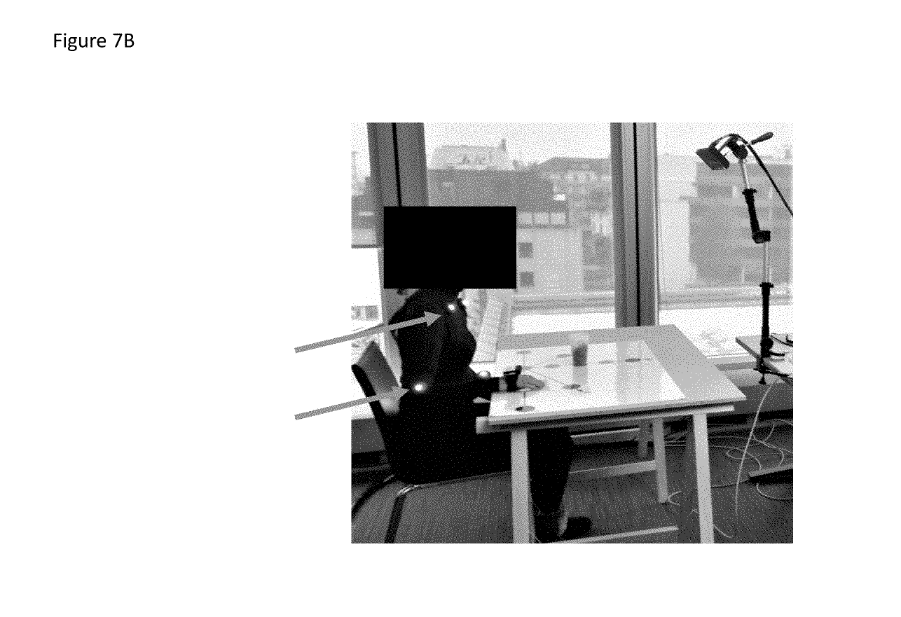

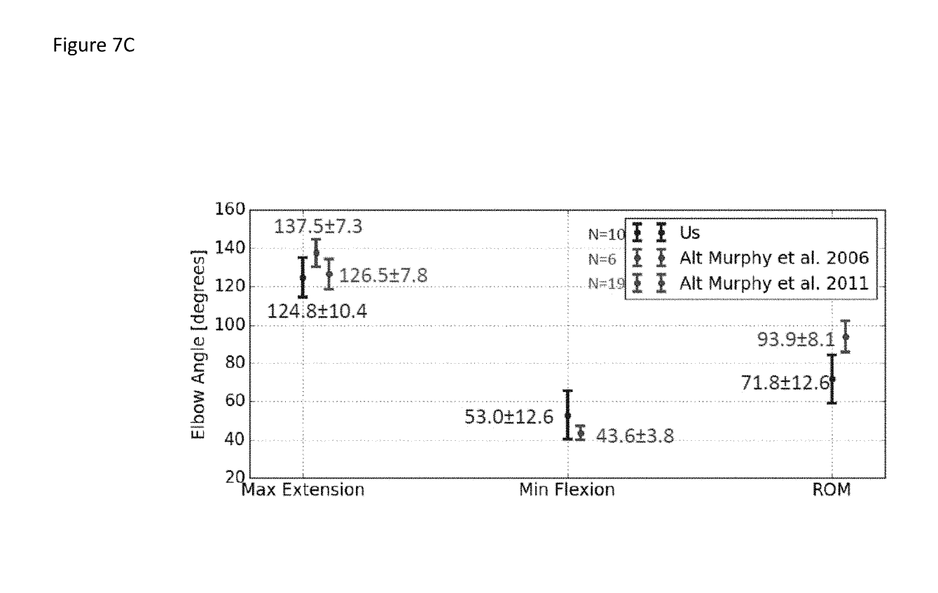

As noted above, a workspace was defined so as to assist the subjects in performing the motions and also measurement of these motions. FIG. 7A shows the workspace, which is similar to that of the workspace of FIG. 4B and of FIG. 6. The blue targets along the directions 1, 3 and 5 were calculated so that the 1st percentile woman would be able to reach to all positions without fully extending the elbow. The other two blue targets (direction 2 and 4) were placed to be on a curved path between them. The purple targets were placed along the midsagittal plane in increments of 10 cm to be used for the exercise verifying at what distance trunk displacement commences. The height of the low platform, 11 cm, was half the length of the forearm of the 1st percentile woman so that it would be still possible to grab objects from the platform without lifting the elbow off the table. The mid- and high-level platform heights were defined as the shoulder height, 23 cm, and the eye-level height, 38 cm, of the 1st percentile woman, respectively.

The chosen task was to take a spoon and put it in the cup, and then take a sugar cube to put in the cup. The last part of the sequence includes bringing the cup back towards oneself to see if this movement is performed at a slower pace than earlier. The weighted plastic cup weighed 150 g to be similar to other research groups. The cup was 5.3 cm and 7.3 cm in diameter at the bottom and top respectively, and 11.7 cm high. For the fine motor task, a 19 cm long metal spoon was used, and a sugar cube of dimensions 3.5×2.3×1.2 cm was used.

The movements of 10 subjects were measured. The subjects were seated so that their feet were firmly on the floor. The chair was positioned so that the subjects' wrists were aligned with the edge of the table when they had 0. shoulder flexion and adduction. The camera was aligned to the mid-sagittal plane of the subject and approximately a meter away, as shown in FIG. 7B (the identifying features of the subject were blocked for privacy).

The end-effector workspace was calculated by finding the maximal position of the wrist marker in three perpendicular directions during the reaching exercise without trunk movement. The results were used to adapt the virtual object grabbing height, and the target positions of the MindMotion™ PRO VR grasp exercise. The start pad was positioned at the same distance away from the body as the green target from the assessment had been (FIG. 7A). The object height was positioned at 95% of the maximal recorded height (direction Zₘₐₓ), and there was one target per Yₘₐₓ, Xₘₐₓ and Xₘᵢₙ directions, also placed at 95% of the maximal recorded distance during the assessment. This movement was chosen for the workspace as trunk displacement should be avoided during acute stroke rehabilitation, and it was shortened by 5% as full extension of the arm without trunk movement is unrealistic in everyday activities. Nine of the subjects completed the adapted VR exercise, and the difference between the wanted displacement and the actual displacement was calculated. The second observed parameter, the trunk displacement, was measured as the mid-point between the two shoulder markers. It was measured during the reaching task to verify that the subjects did not move their trunk, and also during the adapted VR exercise for the same reason. It was also measured during the task placing the cup 50 cm away from the table edge to see that there was trunk displacement when wanted. The last parameter, the elbow ROM (range of movement), was calculated for the drinking task as it was the task which required the highest ROM.

Results

The measurements by the MindMotion™ PRO system were consistent with actual movements of the subjects' hands and of objects grasped therein, showing that the system correctly measurements movement and displacement in space (data not shown). In addition, the measurements of movements made in a virtual environment were consistent with results obtained by other researchers with actual physical objects, as shown in FIG. 7C.

For the measurements in FIG. 7C, the blue data represents the maximal, minimal and range of motion of the elbow measured during the drinking task by the MindMotion™ PRO system. The green and red data represent the elbow angle data measured by Alt Murphy et al. during a drinking task by 6 and 11 healthy subjects in 2006 and 2011 (M. Alt Murphy, K. S. Sunnerhagen, B. Johnels, and C. Willén, “Three-dimensional kinematic motion analysis of a daily activity drinking from a glass: a pilot study,” Journal of NeuroEngineering and Rehabilitation, vol. 3, pp. 18-18, August 2006; M. Alt Murphy, C. Willén, and K. S. Sunnerhagen, “Kinematic variables quantifying upper-extremity performance after stroke during reaching and drinking from a glass,” Neurorehabilitation and Neural Repair, vol. 25, no. 1, pp. 71-80, 2011) respectively. As for the present study, the cup in the 2006 and 2011 studies was placed 30 cm away from the table edge.

FIG. 7C therefore shows elbow displacement and angle as measured in the virtual environment of the MindMotion™ PRO system (blue) and also as measured in a real-world environment in the previously described two separate studies (green—2006 study; red—2011 study). Strikingly, the measured elbow displacements are highly similar in all three studies, indicating that measurements in the virtual environment are highly similar to those in actual physical environments.

System Operation Adjustments

Trunk Involvement—

As noted above, various adjustments to the operation of the MindMotion™ PRO system or other embodiments may be made for various therapeutic effects. In particular, the games may be adjusted to avoid involvement of the trunk, for example during an early time period after brain injury, such as after stroke. Such adjustments may include setting a maximum reach to avoid trunk involvement, such as for example up to 95% of the maximum reach displayed during calibration, up to 90%, up to 85%, up to 80% or any number in between.

Determining Trunk Involvement—

In order to prevent trunk involvement or trunk motions from occurring, it is necessary to at least detect trunk involvement. Such detection may optionally be performed by measuring trunk displacement as the mid-point between the two shoulder markers. As noted above, by “markers” it is optionally meant any type of detection of specific points associated with the joints, whether with active markers or through other types of detection. Preferably, specific markers associated with the shoulders are used for the determination of trunk involvement; optionally the game or virtual environment interactions may then be adjusted to reduce or remove trunk involvement, for example by adjusting the location of the virtual object.

### Example 5—Performance of MindMotion™ PRO with Subject after Stroke

The performance of the MindMotion™ PRO system was tested with a subject who had suffered a stroke. A 60-year-old right-handed (Edinburgh test >95) male ischemic stroke (left paramedian pontine) subject with hemiparesis on the right side (at Day 8: NIHSS=6, Fugl-Meyer Assessment=26/66) was recruited. In addition, 13 age-matched right-handed (Edinburgh test >80) adults (58 9 years; 7 males and 6 females) were also recruited.

All participants performed planar reaching exercises that implied active shoulder and arm movements using the MindMotion™ PRO system that provided embodied two embodied visual feedback: (i) direct mode (arm movements are translated to the ipsilateral an avatar on the computer screen); and (ii) mirror mode (arm movements are translated to the contralateral side of the avatar). These activities may be described as VR mediated mirror visual feedback (VR-MVF). The 16 electrode Electroencephalogram of the participants was recorded from frontal, central and parietal areas, while performing the movements.

The results showed that VR-MVF is likely to influence cortical sub-threshold activity during movement preparation and execution, and results in more balanced activity as observed in MRCPs (Movement-Related Cortical Potentials). Without wishing to be limited by a single hypothesis, VR-MVF may increase cortical excitability around the penumbra of the lesion in a selected group of stroke patients and hence could be used as a therapeutic intervention.

Methods

The previously described stroke survivor was hospitalized on Day 0, assessments were performed on Day 8 (Pre), Day 13 (Post) and after follow-up. The intervention was performed for 3 consecutive days, Day 9, 10 and 11 using MindMotion™ PRO system for neurorehabilitation as previously described with centered-out reaching tasks directly at the bedside at the acute neurorehabilitation center. The participant also received approximately full body physiotherapy approximately 60 min per day.

Experimental Setup

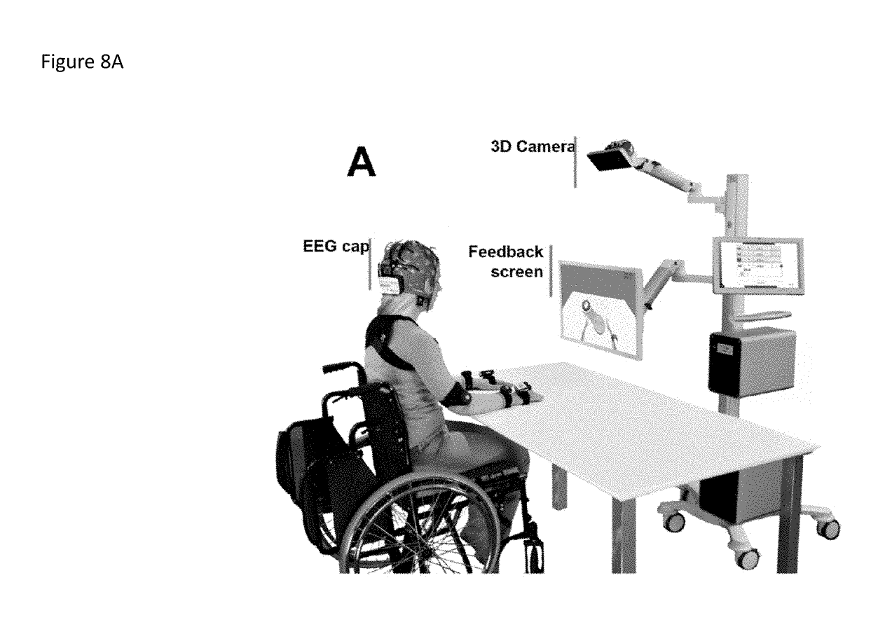

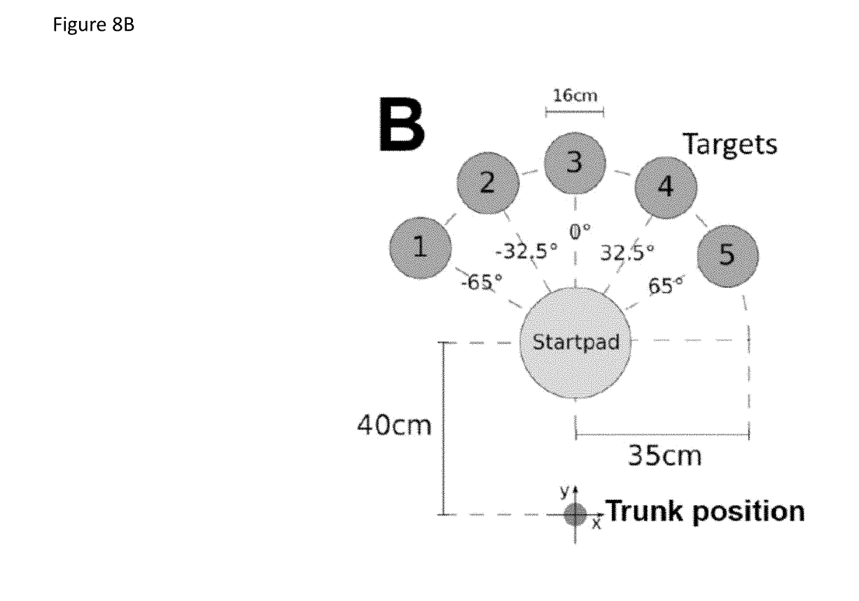

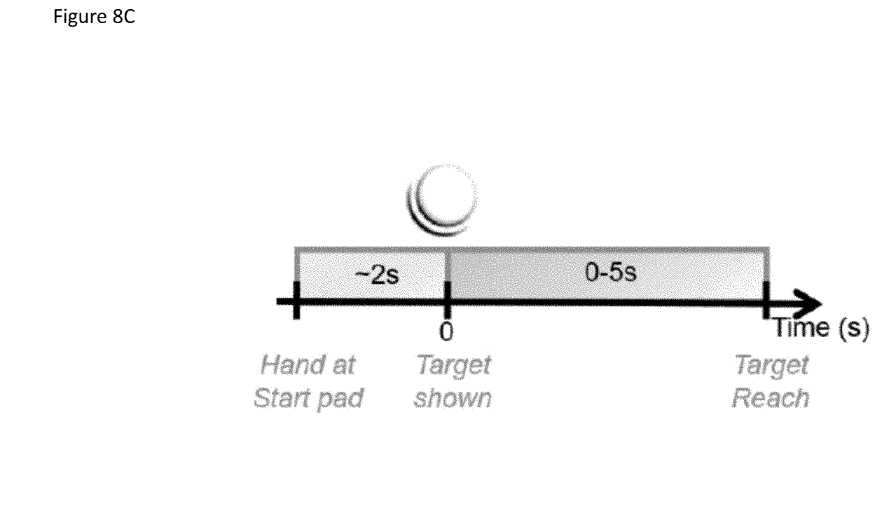

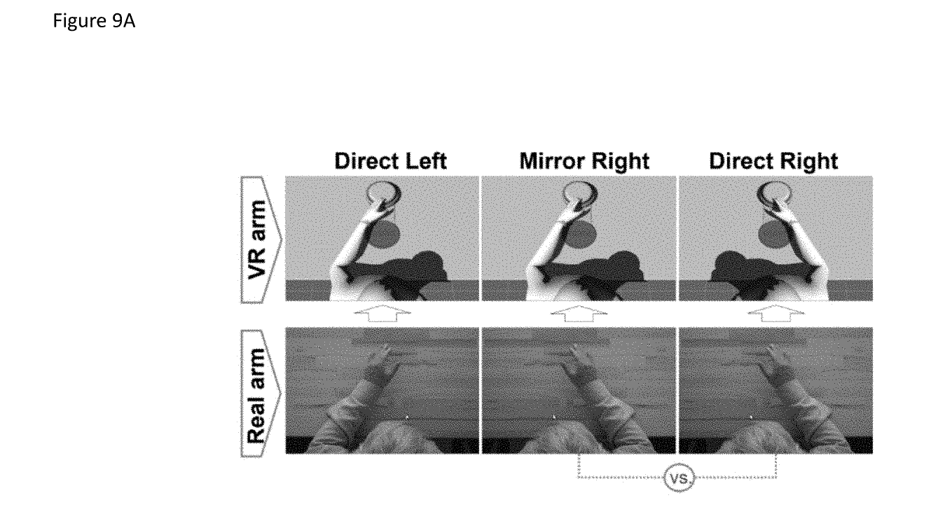

Participants were asked to execute upper limb centered out reaching movements in a 2-dimensional plane (i.e. over a physical table) to five locations randomly presented using the MindMotion™ PRO system as previously described for delivering game-like rehabilitation exercises. The system, shown in FIG. 8A, also included an EEG (electroencephalogram) cap for measuring EEG signals. Such measurements could optionally be used to provide feedback to the participants (not shown). The participants sat in a comfortable chair or wheelchair in front of a table that acted as gravity support as shown. For this implementation, the MindMotion™ PRO system includes of a motion-capture camera (placed approximately 180 cm of above the floor) tracking the upper extremity, a feedback screen (approx. 120 cm) projecting first person perspective of an avatar that reproduced movements of the participant in real time.

In a typical centered-out reach exercise, when ready, the patient places hand on Start pad, and after a random period of time (max 2 s) a target (35 cm distance) is shown in one of the five locations (each 32.5 cm apart) as shown in FIG. 8B. The participant is then allowed to reach to the target location with a speed of their comfort. After about 1 s the target reach the participant is provided a rewarding score that corresponded to the trajectory accuracy. During the movement, the participant was also provided a real-time visual feedback on movement error notification if the hand deviated from the presented trajectory path. The participants were asked to use their left or right arm in executing the reaching task depending on the experimental condition and the feedback was provided either on the same side (Direct) of the avatar or opposite (Mirror). In Mirror condition, the movements of the right and left arms are exchanged over the mid-sagittal plane, while keeping the shoulder internal/external rotation and forearm pronation and supination same. For example, participant's active right arm is mapped onto the virtual left arm, and vice versa.

FIG. 9A shows an example of the motions performed by the healthy participants. All healthy participants performed the centered-out reaching movements under the following conditions as (a) Direct Left, where participants moved the left arm and the avatar reproduced with left arm, (b) Mirror Right, where participants moved the right arm and the avatar reproduced with left arm and (c) Direct Right, where the participants moved the right arm and the avatar reproduced with right arm. Each participant performed 100 repetitions per condition per session that lasted about an hour including pauses. The conditions were presented in random order.

FIG. 9B shows an example of the motions performed by the stroke survivor. Similar to the healthy participants, the stroke survivor also performed the centered out reaching tasks but with the following conditions over 3 days starting from Day 8, where Day 0 was hospitalization: (a) Direct Left (40, 60 and 110 repetitions for Day 8, 9 and 10 respectively) (b) Mirror Left, where the patient moved left arm (i.e. the non-paretic arm) and the avatar reproduced on the right arm (40, 95 and 80 repetitions for Day 1, 2 and 3 respectively) and (c) Direct Right the stroke survivor made an effort to move the right arm (i.e. the paretic arm) with 10, 85 and 95 repetitions over Day 1, 2 and 3 respectively. The order of the conditions were chosen according to the fatigue and the participant's comfort.

Data Collection

Electroencephalogram

From all participants full-band electroencephalogram (fbEEG) data was recorded in 10-20 international system using 16-electrodes (FC3, FCz, FC4, C5, C3, C1, Cz, C2, C4, CP3, CP1, CP2, CP4 and Pz) spanning frontal, central and parietal areas, while performing the centered out task, using g.USBamp (g.tec medical engineering GmbH, Austria) at the sampling rate of 512 Hz. Two additional electrodes were used as ground (AFz) and reference (right earlobe).

Outcome Measures

The stroke participant's impairment was measured using National Institute of Healthy Stroke Scale (NIHSS) (L. Zeltzer, Stroke engine: Assessments: National Institutes of Health Stroke Scale (NIHSS)), Fugl-Meyer upper limb scale (FMUL) (A. R. Fugl-Meyer, L. Jaasko, I. Leyman, S. Olsson, S. Steglind, The poststroke hemiplegic patient. 1. a method for evaluation of physical performance, Scand J Rehabil Med. 7 (1975) 13-31) in motor, sensation and passive joint motion and Frenchay arm test (FAT) (D. T. Wade, R. Langton-Hewer, V. A. Wood, C. E. Skilbeck, H. M. Ismail, The hemiplegic arm after stroke: measurement and recovery, Journal of Neurology, Neurosurgery, and Psychiatry 46 (1983) 521-524) at Day 8, Day 13 and at the follow up day. Additionally, the patient's self-reporting on various aspects of motivation and engagement was recorded.

Data Processing

Electroencephalogram

Firstly, the DC offset of the EEG recordings was reduced by removing the first sample from each electrode data. Then, the data was downsampled to 64 Hz (using zero-phase low-pass 3rd order Butterworth filter fc=24.6 Hz). The data was then band-pass filtered in the range 0.1 1.5 Hz that correspond the spectral content of movement related slow cortical potentials (SCPs) in EEG. The data was then re-referenced to the average activity of C5 and C6.

Grand-Averages

Epochs were extracted from the full recordings using a −2 s to −5 s time window around movement onset at 0 s (FIG. 8C). For each trial the movement onset was detected using the motion capture system as the deviation of hand out of an imaginary start-pad sphere that aligned with the start-pad (i.e., same radius) in the VR environment.

The potential recorded from each electrode, for each epoch, was then corrected for baseline activity, identified at −1.5 s. The trials were rejected if the movement onset was detected too late or multiple movement onsets were detected. Additionally, artifact data that may have resulted due to movement or other external causes that exceeded +/−120 microV at any electrode were rejected from the further analysis. The remaining epochs are then averaged across all healthy participants separately for each condition and electrode. The stroke participant EEG epochs data were treated in a similar way. Due to the reduced number of trials available from the stroke participant in the Direct Right (i.e. paretic arm movements) after the artifact rejection, further analysis was limited to Direct Left and Mirror Left conditions.

Topographic plots of the average activity at each channel during a short time window selected using the time point that corresponded to the maximal difference between the conditions observed using student t-test over C2 electrode data across conditions for pooled healthy participant data and stroke participant data separately.

Results

Healthy Participants

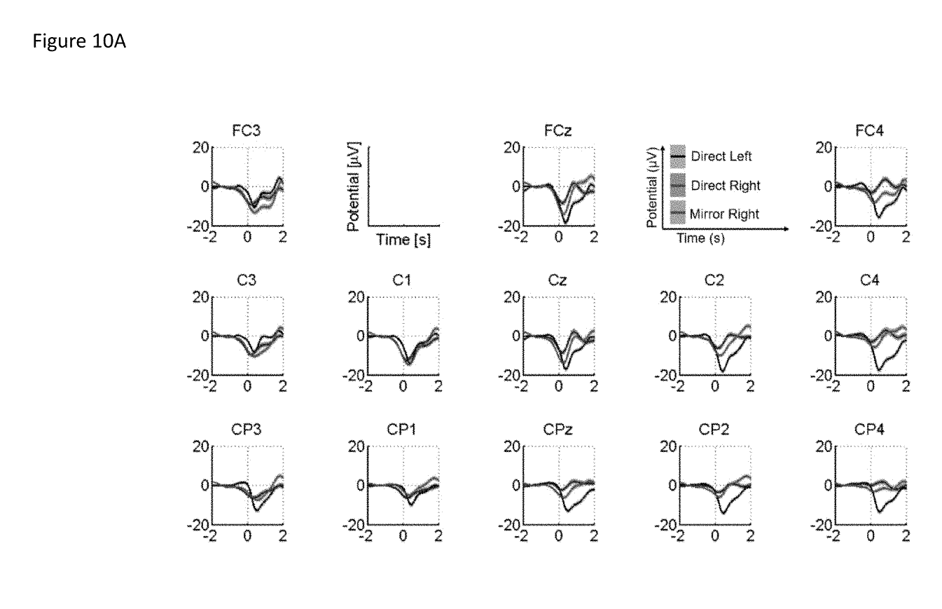

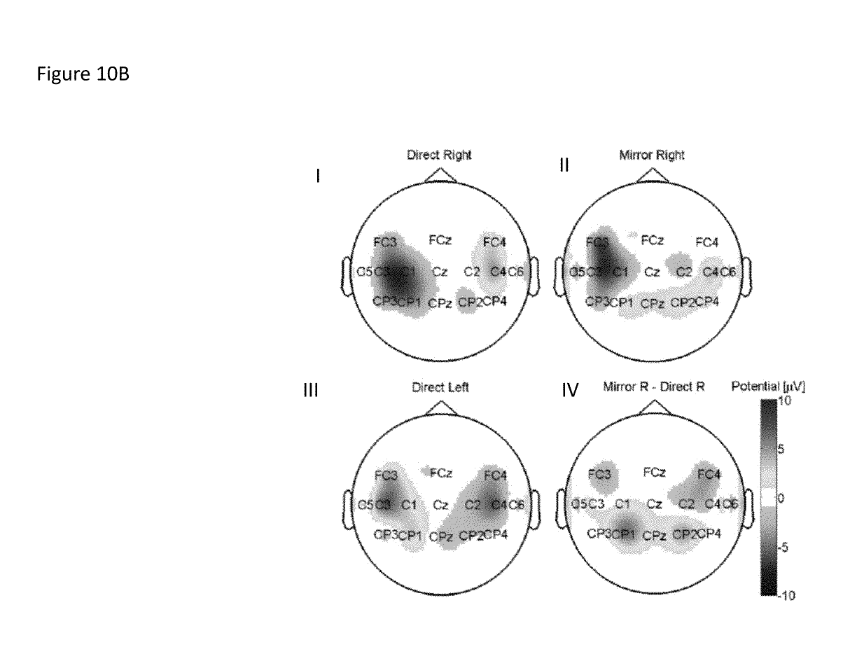

The grand average traces for the pooled data of healthy participants is presented in FIG. 10A for Direct Left, Direct Right and Mirror Right conditions and the difference between Mirror Right and Direct Right. Similar to the widely known lateralized movement related cortical potentials (or readiness potentials), a clear increase in negativity is observed in all the three conditions, however with a difference described as follows. Firstly, as expected Direct Left and Direct Right showed more negative activity on the contralateral side, i.e., right (C2, CP2, FC4, C4 and CP4) and left side electrodes (C1, CP1, FC3, C3 and CP3) respectively. This contralateral negativity is the highest for Direct Left condition with a peak negative activity at C2 electrode (mean+/−SEM: −18.0+/−1.0 microV at 0.4 s), with peak negative activity at Cz electrode of −17.0+/−1.1 microV at 0.4 s and C2 electrode of −14.5+/−0.75 at 0.4 s. Similarly, we also observe highest negativity for Direct Right condition at the C1 electrode (−12+/−1.2 microV) at 0.2 s) with peak negativity at Cz of −14+/−0.8 microV at 0.25 s and −10.0+/−0.7 microV at 0.3 s. Interestingly, the Mirror Right condition showed high negativity trends at C1 (−14.0+/−0.8 at 0.25 s), Cz (−14.8+/−0.8 at 0.30 s), and C2 (−10.0+/−0.7 at 0.30 s).

A window of activity of MRCP for topographic maps was chosen based on maximal differences observed between Mirror Right and Direct Right conditions (t-test, pᵢ0.01). Topographic maps of MRCPs for the all the conditions is shown in FIG. 10B, where the data was obtained using the average of 25 ms MRCP data at time window between 775 ms to 800 ms after the movement onset. The Direct Right condition displayed the well known MRCP negative activity in left side electrodes (at 270 FC3, C3, C1 and CP1; FIG. 10B-I), whereas Direct Left showed negativity right side electrodes (at FC4, C2, C4 and CP2; FIG. 10B-II) confirming the clear lateralization. Interestingly, for the Mirror right condition, the negative activity is also present in the left side electrodes (e.g., FC3, C1 and C3; FIG. 10B-III) including C2 electrode, which is ipsilateral to the active arm. The difference topographic map between Mirror Right and Direct Right is shown in FIG. 10B-IV for the same time window.

One way ANOVA performed at the group level for electrodes separately revealed pᵢ0.01 at electrode sites FC3 (F=7.98), FCz (F=18.7), FC4 (F=33.4), C3 (F=12.6), Cz (F=21.6), C2 (F=29.9), C4 (F=41.7), CPz (F=31.5), CP2 (F=31.3) and CP4 (F=32.4). The pair wise Wilcoxon rank sum test between Direct Right and Direct Left showed significant differences (pᵢ0.01) at electrodes FCz, FC4, Cz, C2, C4, CPz, CP2 and CP4. Similarly, significant differences between Direct Right and Mirror Right was observed at electrodes C1, CP1, CP2 and CP4.

Stroke Participant

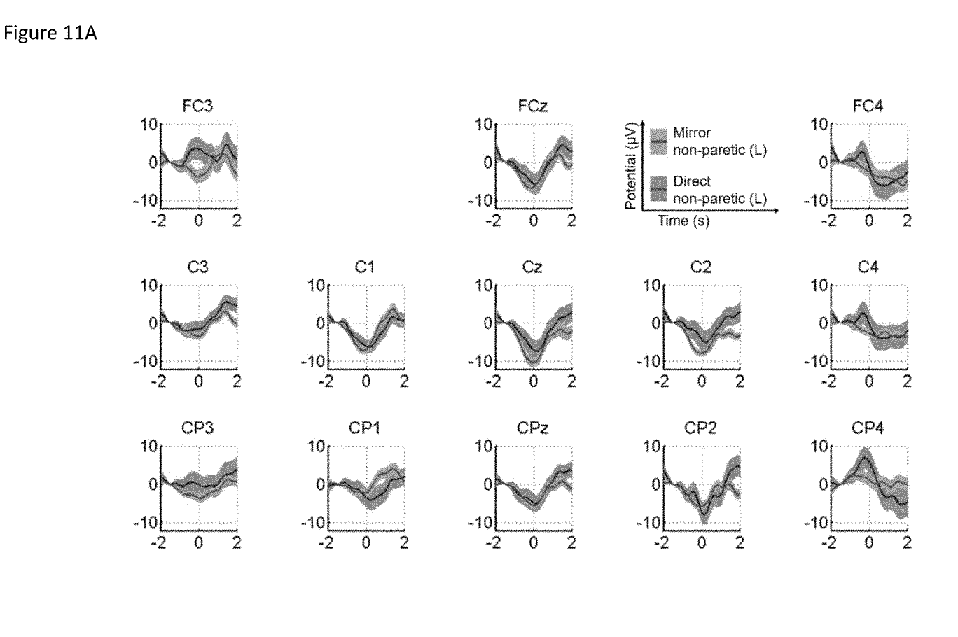

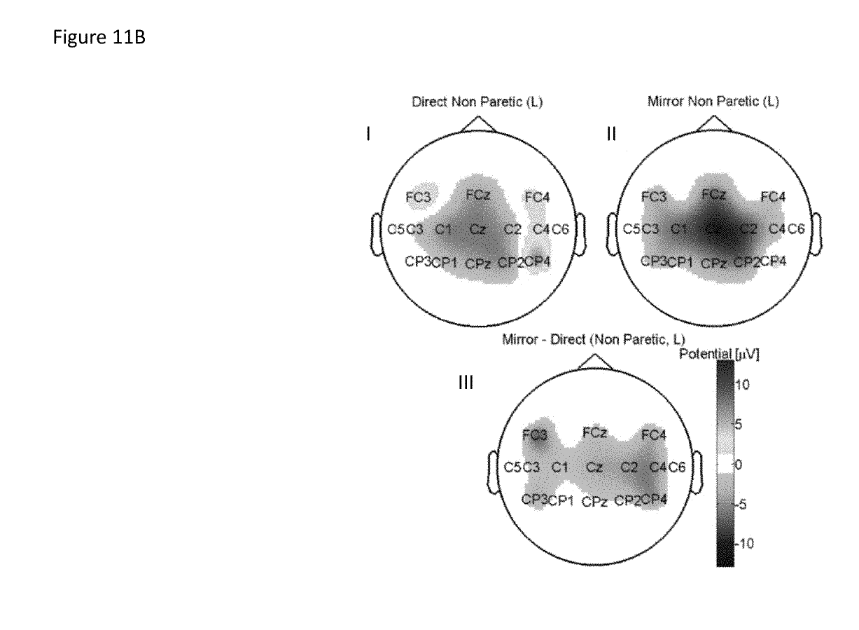

The grand average MRCP data of the stroke participant is presented in FIG. 11A for Direct non-paretic and Motor non-paretic conditions, where the left arm was used for performing the exercise. In Direct Non-paretic condition, a build up of negative potentials similar to movement related/readiness potentials with less clear lateralization with maximum trend negativity observed at electrodes C1 (mean+/−SE: −6.2+/−1.5 microV at 0.15 s), Cz (−7.3+/−3.0 microV at 0.14 s), and C2 (−5.0+/−3.0 microV at 299 0.25 s). Interestingly, the Mirror non-paretic condition elicited similar patterns yet with higher negativity peaks (C1 −7.1+/−1.0 microV, 0.14 s), Cz (−10.4+/−1.2 microV, at 0.15 s) and C2 (−7.8+/−0.8 microV, at 0.05 s). Overall, the latency of the negative peak was earlier than that of the healthy participants.

A window of activity of MRCP for topographic maps was chosen based on maximal differences observed between Mirror Right and Direct Right conditions (t-test, pᵢ0.01). Topographic maps of MRCPs for the all the conditions are shown in FIG. 11B-III, where the data was obtained using the average of 25 ms MRCP data at time window between −225 ms to 200 ms (i.e. before the movement onset). Higher negativity is shown both in ipsi-lesional (FC3 electrode (95% CI [0.758, 13.2])) and contra-lesional side (C2 electrode C2 (95% CI [0.0414, 9.52])) in Mirror non paretic condition compared to Direct non paretic condition.

Table 5 shows the various improvements in the condition of the stroke participant according to various measures. The outcome measures of the stroke participant were measured at Pre and Post VR-sessions and at Follow-up.

TABLE 5

| Assessment | Pre (T1) | Post (T2) |
|---|---|---|
| NIHSS (max: 42) | 6 | 3 |
| FAT (max: 5) | 3 | 3 |
| FMA-UE | | |
| A. Upper extremity synergies (max: 36) | 18 | 24 |
| B. Wrist (max: 10) | 0 | 2 |
| C. Hand (max: 14) | 3 | 2 |
| D. Coordination/Speed (max: 6) | 4 | 5 |
| Motor function total A-D (max: 66) | 26 | 32 |
| Sensation (max: 12) | 8 | 8 |
| Passive joint motion (max: 24) | 22 | 21 |
| Joint pain (max: 24) | 21 | 24 |

The NIHSS score reduced from 6 to 3 post VR-intervention, which corresponds to a mild impairment and hence discharged from the acute neurorehabilitation center. The FAT score did not change from 3 Pre to Post but improved at the Follow-up. Furthermore, the motor impairment of the right arm as measured with FMA-UE in motor function, sensation, passive joint motion and joint pain showed clinically important improvement (=6 points; with main contributions from the synergies of shoulder, elbow and forearm) observed in motor function with at Post compared to Pre VR-intervention (=26 points), which further improved.

### DISCUSSION

The stroke participant showed significant improvement in various measures as described herein. The addition of the EEG cap permits additional feedback to be provided to such participants, such that the method of treatment may be adjusted according to the EEG measurements.

Furthermore, the stroke participant performed a high number of activities with the MindMotion™ PRO system: during the 3 day (Day 9-11) intervention the patient performed 160 Direct Paretic and 150 Mirror non-paretic repetitions, which is generally considered as high intensity during the acute hospitalization period. Pre (Day 8) and Post (Day 13) VR therapy intervention showed an increase in FMA-UE score of 6 points which is above minimal clinically important difference in FMA-UE score. The score further increased at the follow-up. An improvement in the joint pain of 3 points at the post-therapy and further improvement at the follow-up was also observed. By answering a routine questionnaire, the stroke participant reported a high level of concentration, enjoyment and relaxation although increased level of fatigue while performing the VR exercises. Interestingly, he also reported the willingness to continue performing the VR exercises at home and demanded more challenging exercises.

While the invention has been described with respect to a limited number of embodiments, it will be appreciated that many variations, modifications and other applications of the invention may be made, including different combinations of various embodiments and sub-embodiments, even if not specifically described herein.

1. A method with a computationally directed set of movements, the set of movements being directed by a computational system that comprises providing visual direction to the patient and tracking the movements of the patient, the computational system comprising a display, at least one tracking sensor and a plurality of machine instructions for controlling the display to provide the visual direction and for receiving sensor data from the tracking sensor to track the movements of the patient, the method comprising:
displaying a virtual object to the patient;
indicating a movement to be performed with the virtual object;
tracking said movement using the computational system, the tracking comprising obtaining tracking data with a depth camera and an RGB camera of the computational system, obtaining tracking data with a plurality of inertial sensors attached to the patient and an inertial sensor receiver of the computational system, or both; analyzing the tracking data using a tracking engine to identify movements of the patient and generate patient movement data; and analyzing patient movement data using a data analysis layer to generate a display adjustment parameter and indicator adjustment parameter; and
adjusting said displaying according to the display adjustment parameter and said indicating according to the indicator adjustment parameter;
wherein said displaying, indicating and tracking apply a higher degree of therapeutic intensity as compared to a standard of care rehabilitative measure, wherein said higher degree of therapeutic intensity comprises increasing an amount of time the patient spends during each therapeutic session, increasing a number of exercises that the patient performs during said session within a specific time frame, or both.

2. (canceled)

3. The method of claim 1, wherein the computational system comprises a MindMotion™ PRO system.

4. The method of claim 1, wherein said standard of care rehabilitative measure comprises GRASP.

5. The method of claim 4, wherein said standard of care rehabilitative measure comprises at least one of reduced joint pain and improved motor function.

6. The method of claim 5, further comprising providing an improvement from baseline in upper extremity motor function measured by the Fugl-Meyer Assessment for Upper Extremity (FMA-UE) and/or its subscales as compared to said standard of care rehabilitative measure.

7. The method of claim 6, further comprising providing an improvement from baseline in upper extremity motor ability measured by the streamlined Wolf Motor Function Test (sWMFT) score as compared to said standard of care rehabilitative measure.

8. The method of claim 7, further comprising providing an improvement from baseline in self-care ability measured by the Barthel index (BI) as compared to said standard of care rehabilitative measure.

9. The method of claim 8, further comprising providing an improvement from baseline in functional independence measured by the Modified Ranking Scale (MRS) and/or associated disability-adjusted life year (DALY) as compared to said standard of care rehabilitative measure.

10. The method of claim 9, further comprising providing an improvement from baseline in the general health status as measured by the Stroke Impact scale (SIS) as compared to said standard of care rehabilitative measure.

11. The method of claim 10, further comprising providing an improvement from baseline in the severity of stroke symptoms as measured by the NIH stroke scale (NIHSS) as compared to said standard of care rehabilitative measure.

12. The method of claim 11, further comprising providing an improvement from baseline in arm function in daily activities as measured by the Motor Activity Log (MAL) as compared to said standard of care rehabilitative measure.

13. The method of claim 12, further comprising providing an improvement in motivation measured by the Intrinsic Motivation Index (IMI) as compared to said standard of care rehabilitative measure.

14. The method of claim 13, further comprising providing reduced therapist time spent administrating rehabilitation exercises as compared to said standard of care rehabilitative measure.

15. The method of claim 14, further comprising providing an improvement from baseline in upper extremity muscle strength measured by the Medical research Council Scale (MRC) as compared to said standard of care rehabilitative measure.

16. The method of claim 15, wherein said muscle strength comprises one or more of strength for shoulder elevation, elbow flexion/extension, forearm pronation/supination and wrist extension/flexion.

17. (canceled)

18. The method of claim 1, further comprising providing an increased rehabilitation dose as measured by the duration of the rehabilitation session without planned rest periods as compared to said standard of care rehabilitative measure.

19. The method of claim 1, comprising performing the method during an acute period following a neurological trauma.

20. The method of claim 19, wherein said neurological trauma comprises at least one of stroke and head injury.

21. The method of claim 1, wherein the virtual object is displayed to the patient in an AR (augmented reality) or VR (virtual reality) environment.

22. The method of claim 21, further comprising determining a location of the virtual object in the AR or VR environment to avoid trunk involvement in a movement by the patient.

23. The method of claim 22, further comprising performing a calibration to determine a maximum reach of the patient and performing said determining said location according to a maximum of a range of 80-95% distance of said maximum reach of the patient.

24. The method of claim 23, wherein said maximum distance is 95% of said maximum reach of the patient.

25. The method of claim 23, further comprising measuring an extent of trunk involvement according to movement of shoulders of the patient.

26. The method of claim 1, further comprising measuring EEG signals of the patient during said tracking said movement by the patient.

27. The method of claim 26, further comprising providing feedback to the patient according to said EEG signals.

28. The method of claim 1, further comprising providing feedback to the patient through a visual display of a mirror avatar.

29. (canceled)

30. The method of claim 1, further comprising:
assessing an ability of the patient to perform a movement with a physical object;
displaying a virtual object to the patient according to said assessing;
indicating a movement to be performed by the patient with the virtual object, determined according to said assessing;
tracking said movement according to said tracking data; and
adjusting said displaying and said indicating according to said tracking.

31. (canceled)

32. The method of claim 30, wherein the virtual object is displayed to the patient in an AR (augmented reality) or VR (virtual reality) environment.

33. (canceled)

34. (canceled)

35. (canceled)

36. The method of claim 30, further comprising:
assigning a visual indicator to the virtual object, the visual indicator communicating to the user to correct a trajectory.

37. (canceled)
